(19) United States
(12) Patent Application Publication
(10) Pub. No.: US 2009/0247520 A1
(43) Pub. Date: Oct. 1, 2009

(54) INHIBITORS OF ANTIGEN RECEPTOR-INDUCED NF-kappa B ACTIVATION

(75) Inventors: John C. REED, La Jolla, CA (US); Ranxin SHI, La Jolla, CA (US); John R. Cashman, La Jolla, CA (US); Karl J. OKOLOTOWICZ, La Jolla, CA (US); Daniel RE, Cologne (DE)

Correspondence Address: TechLaw LLP;10755 Scripps Poway Parkway, Suite 465, San Diego, CA, 92131, US

(73) Assignees: The Burnham Institute for Medical Research, La Jolla, CA (US); The Human Biomolecular Research Institute, San Diego, CA (US)

(21) Appl. No.: 12/412,091

(22) Filed: Mar. 26, 2009

Related U.S. Application Data
(60) Provisional application No. 61/040,069, filed on Mar. 27, 2008.

(51) Int. Cl. A61K 31/4184; C07D 413/04; C07D 235/04; A61K 31/5377; A61P 43/00

(52) U.S. Cl. 514/234.5; 544/139; 548/309.7; 514/394

(57) ABSTRACT

A method to identify selective inhibitors of antigen receptor-mediated NF-κB activation is provided, as well as compositions having one or more of those inhibitors and methods of using those inhibitors.

### RELATED APPLICATIONS

The present application claims priority to the U.S. Provisional Application Ser. No. 61/040,069, filed on Mar. 27, 2008, by John C. Reed et al., and entitled “INHIBITORS OF ANTIGEN RECEPTOR-INDUCED NF-κB ACTIVATION”, the entire disclosure of which is incorporated by reference herein.

### STATEMENT OF GOVERNMENT RIGHTS

The invention was supported, at least in part, by a grant from the Government of the United States of America (National Institutes of Health (NIH) grant 1 X01 MH077633-01). The Government has certain rights to the invention.

### FIELD OF THE INVENTION

The present invention is in the field of pharmaceuticals, and particularly in the field of compounds that selectively inhibit antigen receptor-mediated NF-κB activation, high-throughput assays for identifying the same, and methods of using the same.

### BACKGROUND OF THE DISCLOSURE

Members of the nuclear factor-kappa B (NF-κB) family of transcription factors play crucial roles in the control of many physiological and pathological processes, including host-defense, immune responses, inflammation, and cancer. In mammals, at least nine pathways leading to NF-κB activation have been elucidated, including; (i) a “classical” pathway induced by Tumor Necrosis Factor (TNF) and many TNF-family cytokine receptors, involving degradation of Inhibitor of NF-κB-alpha (IκB-a) and release of p65-50 NF-κB heterodimers; (ii) an “alternative” pathway activated by selected TNF-family receptors (e.g. CD40, Lymphotoxin-β Receptor, BAFF Receptor) involving p100 NF-κB2 proteolytic processing to generate p52, a preferred heterodimerization partner of NF-κB-family member RelB; (iii) the Toll-like receptor pathway for NF-κB induction, involving TIR domain-containing adapters and IRAK-family protein kinases; (iv) a pathway activated by exogenous RNA, involving Helicard/Mda5, RIG-1 and mitochondrial protein MAVS, of importance for host defenses against viruses; (v) a DNA damage pathway involving PIDD, a target of p53; (vi) NLR/NOD-family proteins, cytosolic proteins that oligomerize in response to microbial-derived molecules, forming NF-κB-activating protein complexes; (vii) Inhibitors of Apoptosis Proteins (IAPs), including X-linked Inhibitor of Apoptosis Protein (XIAP) which binds TAB/TAK complexes to stimulate NF-κB activation; (viii) oncogenic fusion proteins comprised of portions of cIAP2 and mucosa-associated lymphoid tissue-1 (MALT1), which drive NF-κB activation via interactions with TRAF2 and TRAF6 and a pathway induced by B-cell and T-cell antigen receptors, involving a cascade of interacting proteins that includes caspase recruitment domain-containing membrane-associated guanylate kinase protein-1 (CARMA1, Bimp3), Bcl-10, and MALT (Paracaspase), Caspase-8, and other proteins. The core event upon which these nine NF-κB activation pathways converge is activation of Inhibitor of KB Kinases (IKKs), typically comprised of a complex of IKK-α, IKK-β, and the scaffold protein, IKK-γ/NEMO. In all but the “alternative” NF-κB pathway, IKK activation results in phosphorylation of an IκB-α, targeting this protein for ubiquitination and proteasome-dependent destruction, thus releasing p65/p50 NF-κB heterodimers from IκB-α in the cytosol, and allowing their translocation into nucleus where they initiate transcription of various target genes.

The NF-κB pathway activated by antigen receptors is critical for acquired (as opposed to innate) immunity, contributing to T- and B-lymphocyte activation, proliferation, survival, and effector functions. Dysregulated NF-κB activation in lymphocytes can contribute to development of autoimmunity, chronic inflammation, and lymphoid malignancy. The NF-κB activation pathway linked to antigen receptors involves a cascade of adapter and signal transducing proteins that minimally include a CARMA family protein, Bcl-10, MALT (Paracaspase), TRAF6, Ubc13, and Caspase-8. Formation of this complex is initiated by PKC-mediated phosphorylation of CARMA proteins. In T and B cells, this pathway is initiated by Protein Kinase C(PKC)-theta and PKC-beta, respectively, leading ultimately to IKK activation through a mechanism possibly involving lysine 63-linked polyubiquitination of IKK-γ. Thus, the antigen receptor pathway for NF-κB activation is initiated and concluded by activation of protein kinases—namely, PKCs and IKKs, respectively. Contributions to the PKC-activated NF-κB activation mechanism are also made by Caspase-8, apparently forming heterodimers with c-FLIP and inducing proteolytic processing of c-FLIP. Although IKKs represent logical targets for potential drug discovery, chemical inhibitors of IKKs suppress all known NF-κB activation pathways, and thus lack the selectivity required to inhibit lymphocyte responses without simultaneously interfering with innate immunity and thus creating broad immunosuppression with considerable risk of infection.

### SUMMARY OF THE INVENTION

Disclosed herein is a compound of Formula I or Formula II

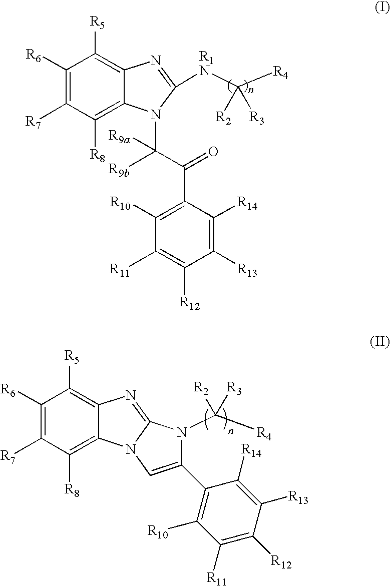

or a pharmaceutically acceptable salt or prodrug thereof, where

R₁ is selected from the group consisting of hydrogen, alkyl, cycloalkyl, and aryl;

R₂, R₃, R₉ₐ, and R_{9b} are each independently selected from the group consisting of hydrogen, alkyl, cycloalkyl, and aryl;

R₄ is selected from the group consisting of hydrogen, —OR₂₀, —SR₂₀, —C(O)OR₂₀, and —C(O)R₂₀;

R₅-R₈ and R₁₀-R₁₄ are each independently selected from the group consisting of hydrogen, —OR₂₀, —SR₂₀, —C(O)OR₂₀, —C(O)R₂₀, alkyl, cycloalkyl, and aryl;

R₂₀ is selected from the group consisting of hydrogen, alkyl, cycloalkyl, and aryl; and

n is an integer between 0-10,

and pharmaceutical compositions comprising the same.

Also disclosed herein are methods of identifying a compound that selectively inhibit antigen receptor-mediated NF-κB activation comprising: (a) providing an aqueous solution comprising a cell transfected with a reporter gene driven by a NF-κB responsive promoter; (b) adding to the solution a test compound; (c) adding to the solution an NF-κB inducing stimulus; and (d) determining whether the test compound reduces the cell response to the stimulus. In some embodiments, the test compound is a compound of Formula I or Formula II, as described herein.

In addition, disclosed herein are methods of selectively inhibiting antigen receptor-mediated NF-κB activation in a cell comprising contacting the cell with a compound of Formula I or Formula II, as described herein. In some embodiments, the cell is in a subject in need of such inhibiting.

Further, disclosed herein are methods of treating a disease associated with antigen receptor-mediated NF-κB activation in a subject comprising identifying a subject in need thereof and administering to the subject, or contacting the subject with, a compound of Formula I or Formula II, as described herein.

### BRIEF DESCRIPTION OF THE DRAWINGS

FIG. 1 is a flow chart of screening and counter-screenings for selective inhibitors of antigen-receptor induced NF-κB pathway. A total of 114,889 compounds were screened. From a screen of 53,280 commercially available compounds (left column), 519 hits were obtained, of which 248 reconfirmed using the same primary screening assay. Next, the same HEK293-NF-κB reporter gene cell line was stimulated with TNF, leaving 46 compounds that failed to inhibit. Four of these compounds showed cytotoxic activity, leaving 42 compounds, of which 11 showed activity when fresh stocks were ordered and tested. From these 11 active compounds, one hit, CID-2858522, inhibited PMA/Ionomycin-induced production of cytokine IL-8 by the HEK293 cell line. CID-2858522 was then characterized (right column). PDB was substituted for PMA to confirm suppression of an alternative PKC activator. Pathway selectivity was assessed using panels of cell lines, starting with HEK293 cells in which each of the remaining NF-κB activation pathways was stimulated, showing inhibition only of PMA/Ionomycin-induced NF-κB reporter gene activity (panel 1). This was followed by a panel of secondary assays using various cell lines or primary cultured splenocytes (panel 2), measuring various end-points, which included NF-κB luciferase reporter gene activity (“Luc”), cytokine secretion, and 3H-Thymidine (3H-TdR), following stimulation with various inducers of specific upstream activators of NF-κB signaling, including agonists of TCRs (anti-CD3/CD28 for Jurkat, splenocytes), BCRs (anti-IgM for splenocytes), TLRs (LPS for THP.1 monocytes), TNFRs that signal through the “alternative” pathway (LTβR antibody for HeLa cells), and NLRs (γTriDAP for activating NOD1 and muramyl-dipeptide [MDP] for activating NOD2 in McF-7 breast cancer and THP.1 monocytes respectively). CID-285822 inhibited only anti-CD3/CD28-stimulated and anti-IgM-stimulated splenocytes. Various in vitro kinase assays were employed, showing no inhibition, followed by a kinome screen using competitive displacement of ATP.

FIG. 2 shows that CID-2858522 inhibits NFκB activation and IL-8 production induced by PMA/Ion and PDBu. (A) Structures of two hit compounds, CID-2858522 (left) and CID-2998237 (right) are shown; (B, C) 293-NF-κB-luc cells were pretreated for 2 hrs with various concentration of either CID-2858522 (B) or CID-29982387 (C) and then stimulated with TNF (10 ng/mL) or PMA/lonomycin (10 ng/mL; 5 ng/mL) for 16 h. Luciferase activity was measured and data were expressed as a percentage relative to control treatment with DMSO only (mean±SD; n=3). (D) 293-NF-κB-luc cells were pretreated for 2 h with various concentrations of CID-2858522 or CID-2998237, then stimulated with PMA/lonomycin for 16 h. IL-8 release into the medium was measured, expressing data as a percentage relative to control cultures treated with DMSO (mean±SD; n=3). (E) 293-NF-κB-luc cells were pretreated with CID-2858522 and then stimulated with PDBu for 16 h, IL-8 production and NF-κB luciferase activity were measured as above. (F) 293-NF-κB-luc cells were pretreated for 2 h with various concentrations of CID-2858522 or a PKC inhibitor (Bisindolylmaleimide I) at 1 μM followed by PMA/Ionomycin (10 ng/mL) stimulation for 2 h, then p65-DNA-binding activity was measured in nuclear extracts (10 μg protein), expressing data as fold-increase relative to unstimulated cells (mean±SD; n=3).

FIG. 3 shows that CID-2858522 does not inhibit NFκB pathway induced by other NFκB inducers. FIGS. 3 (A-F) HEK293-NF-κB-luc cells were pretreated with CID-2858522 (4 μM), IKK inhibitor, BMS-335541 (10 μM), or PKC inhibitor, Bisindolylmaleimide I (1 μM) for 2 hrs and then transfected with plasmids encoding CD4/TLR4 (A), CD40 (B), NOD1 (C), NOD2 (D), cIAP2-MALT1 (E), or XIAP and TAB (F), or pcDNA as control (compounds were not removed). After 48 hrs, luciferase activity was measured expressing data as fold increase relative to unstimulated cells (mean±SD; n=3). (G-H) HEK293-NF-κB-luc cells were pretreated with CID-2858522 (4 μM), or IKK inhibitor, BMS-335541 (10 μM) for 2 hrs and then cultured with 2 μM doxorubicin (G) or 16 μM retinoic acid (H). After 48 hrs, luciferase activity was measured as above.

FIG. 4 shows that CID-2858522 inhibits IL-2 production induced by anti-cd3/cd28 or PMA/Ion in Jurkat cells. FIG. 4A: Jurkat T cells were treated by anti-cd3/anti-cd28/anti-mouse IgG (6 μg/mL) or PMA/Ion (10 ng/mL) for 24 h, IL-2 production in medium was measured by ELISA kit. FIG. 4B-E: Jurkat T cells were pretreated by IKK inhibitor (B), PKC inhibitor (C), CID-2858522 (D) for 2 h and then treated by anti-cd3/anti-cd28/anti-mouse IgG (6 μg/mL) or PMA/Ion (10 ng/mL) for 24 h, IL-2 production in medium was measured by ELISA kit; E, Jurkat cells were treated by CID-2858522, PKC inhibitor and IKK inhibitor for 24 h, cell viability was measured by ATPlite kit.

FIG. 5 shows that CID-2858522 inhibits anti-IgM-induced NF-κB activation and proliferation of β-lymphocytes. (A) Isolated mouse primary splenocytes were cultured with anti-CD3/anti-CD28 (0.3 μg/mL each) or anti-IgM (3 μg/mL) for 48 h, then 1 μCi 3H-Thymidine was added for 12 h and incorporation into DNA was measured, expressing data as fold increase above unstimulated cells (mean±SD; n=3). (B-D) Primary splenocytes were pretreated for 2 h with IKK inhibitor, BMS-335541 (B), PKC inhibitor, Bisindolylmaleimide I (C) or CID-2858522 (D), then stimulated with anti-CD3/anti-CD28 (0.3 μg/mL) or anti-IgM (3 μg/mL) for 48 h. 1 μCi 3H-Thymidine was added for 12 h and incorporation into DNA was measured, expressing data as percent inhibition relative to control cells treated with DMSO (mean±SD; n=3). (E) Human CLL B cell samples (n=3) were cultured for 12 hrs with compound CID-2858522 or inactive analog MLS-0292123, then stimulated with biotin anti-IgM (10 μg/mL) for 24 hrs. Levels of TRAF1 and β-actin were assessed by immunoblotting, quantified by densitometry and TRAF1 results reported relative to control cells, after normalization for β-actin (mean±SD). (F) Human CLL B-cells were treated with various concentrations of CID-285822 or its inactive analog, MLS-0292123, for 12 h followed by biotin-anti-IgM (10 μg/mL) and avidin (10 μg/mL) for 2 h. Then nuclear extracts were prepared and p65-DNA-binding activity was measured (mean±SD; n=3).

FIG. 6 shows that CID-2858522 does not inhibit IKK or PKC kinase activity. CID-2858522 (8 μM) were tested in in vitro IKK beta (A), PKC beta (β) or PKC theta (C) kinase assays using kits. STS (0.5 μM), IKK inhibitor (10 μM) and PKC inhibitor (1 μM) were used as positive controls.

FIG. 7. CID-285852 inhibits IKKβ phosphorylation induced by PKC activators. (A) HEK293 cells were cultured in 0.5% FBS medium for 24 h and then treated with CID-2858522 (4 μM) or PKC inhibitor, Bisindolylmaleimide I (1 μM) for 2 hrs followed by PMA/Ionomycin (10 ng/mL; 5 ng/mL) for 2 h. Cell lysates were subjected to immunoprecipitation using anti-CARMA1 antibody and analyzed by immunoblotting with anti-phospho-CARMA1 antibody or anti-CARMA1 antibody. (B-E) HEK293 cells were transfected with plasmids encoding myc-CARMA1 in combination with plasmids encoding various other proteins including Caspase-8 (cys287ala) (B), HA-IKK-γ (C), HA-TAK1 (D), and TRAF6 (E). After 36 h. cells were cultured in 0.5% FBS medium for 12 h and then treated with CID-2858522 (4 μM) or Bisindolylmaleimide I (1 μM) for 2 hrs, followed by PMA/Ionomycin treatment (10 ng/mL; 5 ng/mL) for 2 hrs. Cell lysates were immunoprecipated using anti-myc antibody and analyzed by immunoblotting using anti-Caspase8 (B), anti-HA (C,D), or anti-TRAF6 (E) antibodies. (F) HEK293 cells were treated with CID-2858522 (4 μM) or PKC inhibitor (1 μM) followed by PMA/Ionomycin (10 ng/mL; 5 ng/mL) treatment for 2 hrs. Cell lysates were normalized for protein content and analyzed by immunoblotting using anti-FLIP and anti-alpha-tubulin antibodies. (G) HEK293 cells were cultured in 0.5% FBS medium for 24 hrs, then treated with CID-2858522 (4 μM) or its inactive analog, MLS-0292123, (4 μM), or PKC inhibitor (1 μM), followed by treatment for 5 min with PMA/Ionomycin (10 ng/mL) or TNF (10 ng/mL). Cell lysates were immunoprecipitated using anti-IKK-β and analyzed by immunoblotting using anti-phospho-IKK-β antibody or anti-IKK-β antibody (as loading control). Approximate molecular weights of all proteins are indicated in kiloDaltons.

### DETAILED DESCRIPTION OF THE EMBODIMENTS

The present invention provides a chemical biology strategy for identification of chemical compounds that selectively inhibit antigen receptor-mediated NF-κB activation. Described herein are also 2-aminobenzimidazole compounds that inhibit between PKCs and IKKs without blocking other NF-κB activation pathways. These compounds thus provide unique research tools for interrogating the PKC-initiated and antigen receptor-initiated pathways for NF-κB induction. The compounds also represent pathway-selective drugs with utility for autoimmunity and some types of lymphoid malignancies.

### DEFINITIONS

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound disclosed herein may display superior activity compared with the other. When required, separation of the racemic material can be achieved by HPLC using a chiral column or by a resolution using a resolving agent such as camphonic chloride. A chiral compound of Formula I or Formula II may also be directly synthesized using a chiral catalyst or a chiral ligand.

“Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the methods of the present invention. Such organisms include, but are not limited to, mammals such as humans. In the context of the invention, the term “subject” generally refers to an individual who will receive or who has received treatment (e.g., administration of a compound of the invention, and optionally one or more anticancer agents) for cancer.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., non-peroxide oxy (—O—), thio (—S—), carbonyl (—C(═O)—), carboxy (—C(═O)—), imine (C═NH), sulfonyl (SO) or sulfoxide (SO₂).

Specific and preferred values listed below for radicals, substituents, and ranges, are for illustration only; they do not exclude other defined values or other values within defined ranges for the radicals and substituents

“Alkyl” refers to a C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (1-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃

The alkyl can optionally be substituted with one or more alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. The alkyl can optionally be interrupted with one or more non-peroxide oxy (—O—), thio (—S—), carbonyl (—C(═O)—), carboxy (—C(═O)O—), sulfonyl (SO) or sulfoxide (SO₂). Additionally, the alkyl can optionally be at least partially unsaturated, thereby providing an alkenyl.

“Alkenyl” refers to a C₂-C₁₈ hydrocarbon containing normal, secondary, tertiary or cyclic carbon atoms with at least one site of unsaturation, i.e., a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂ CH₂CH₂CH₂CH═CH₂).

The alkenyl can optionally be substituted with one or more alkyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylidenyl” refers to a C₁-C₁₈ hydrocarbon containing normal, secondary, tertiary or cyclic carbon atoms. Examples are methylidenyl (═CH₂), ethylidenyl (═CHCH₃), 1-propylidenyl (═CHCH₂CH₃), 2-propylidenyl (═C(CH₃)₂), 1-butylidenyl (═CHCH₂CH₂CH₃), 2-methyl-1-propylidenyl (═CHCH(CH₃)₂), 2-butylidenyl (═C(CH₃)CH₂CH₃), 1-pentyl (═CHCH₂CH₂CH₂CH₃), 2-pentylidenyl (═C(CH₃)CH₂CH₂CH₃), 3-pentylidenyl (═C(CH₂CH₃)₂), 3-methyl-2-butylidenyl (═C(CH₃)CH(CH₃)₂), 3-methyl-1-butylidenyl (═CHCH₂CH(CH₃)₂), 2-methyl-1-butylidenyl (═CHCH(CH₃)CH₂CH₃), 1-hexylidenyl (═CHCH₂CH₂CH₂CH₂CH₃), 2-hexylidenyl (═C(CH₃)CH₂CH₂CH₂CH₃), 3-hexylidenyl (═C(CH₂CH₃)(CH₂CH₂CH₃)), 3-methyl-2-pentylidenyl (═C(CH₃)CH(CH₃)CH₂CH₃), 4-methyl-2-pentylidenyl (═C(CH₃)CH₂CH(CH₃)₂), 2-methyl-3-pentylidenyl (═C(CH₂CH₃)CH(CH₃)₂), and 3,3-dimethyl-2-butylidenyl (═C(CH₃)C(CH₃)₃.

The alkylidenyl can optionally be substituted with one or more alkyl, alkenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkenylidenyl” refers to a C₂-C₂ hydrocarbon containing normal, secondary, tertiary or cyclic carbon atoms with at least one site of unsaturation, i.e., a carbon-carbon, sp² double bond. Examples include, but are not limited to: allylidenyl (═CHCH═CH₂), and 5-hexenylidenyl (═CHCH₂CH₂CH₂CH═CH₂).

The alkenylidenyl can optionally be substituted with one or more alkyl, alkenyl, alkylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The alkylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylene can optionally be interrupted with one or more non-peroxide oxy (—O—), thio (—S—), carbonyl (—C(═O)—), carboxy (—C(═O)O—), sulfonyl (SO) or sulfoxide (SO₂). Moreover, the alkylene can optionally be at least partially unsaturated, thereby providing an alkenylene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

The alkenylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, The alkenylene can optionally be interrupted with one or more non-peroxide oxy (—O—), thio (—S—), carbonyl (—C(═O)—), carboxy (—C(═O)O—), sulfonyl (SO) or sulfoxide (SO₂).

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl, alkylidenyl, alkenylidenyl, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkenyl, alkoxy, halo, haloalkyl, hydroxy, hydroxyalkyl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkoxy, halo, haloalkyl, hydroxy, hydroxyalkyl, aryl, heteroaryl, heterocycle, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkoxy, halo, haloalkyl, hydroxy, hydroxyalkyl, aryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkoxy, halo, haloalkyl, hydroxy, hydroxyalkyl, aryl, heteroaryl, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each Rx and R^{y} are independently H, alkyl, aryl, heteroaryl, heterocycle, cycloalkyl or hydroxyl.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino [3,2,1-jk] carbazol-3-ium iodide.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH-]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “imino” refers to —C═NH.

The term “nitro” refers to —NO₂.

The term “trifluoromethyl” refers to —CF₃.

The term “trifluoromethoxy” refers to —OCF₃.

The term “cyano” refers to —CN.

The term “hydroxy” or “hydroxyl” refers to —OH.

The term “oxy” refers to —O—.

The term “thio” refers to —S—.

The term “thioxo” refers to (═S).

The term “keto” refers to (═O).

As used herein, “nucleic acid base” refers to a nitrogenous base that is planar, aromatic and heterocyclic. They are typically derivatives of either purine or pymidine. Suitable nucleic acid bases include, e.g., purine, pymidine, adenine, guanine, cytosine, uracil, and thymine.

The nucleic acid base can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in an claim of the invention, the total number will be determined as set forth above.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

Obviously, numerous modifications and variations of the present invention are possible in light of the above teachings. It is therefore to be understood that within the scope of the appended claims, the invention may be practiced otherwise than as specifically described herein.

### Compounds of the Invention

Thus, in one aspect, disclosed herein are compounds of Formula I or Formula II

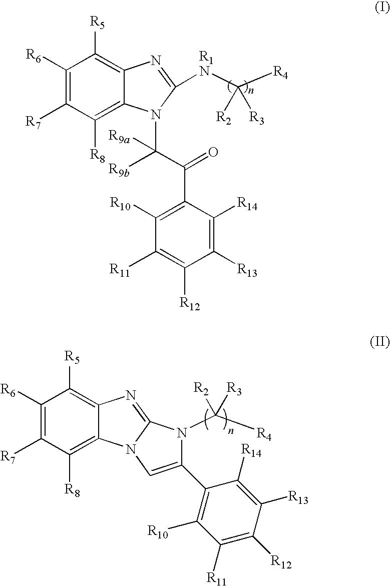

or a pharmaceutically acceptable salt or prodrug thereof, where

R₁ is selected from the group consisting of hydrogen, alkyl, cycloalkyl, and aryl;

R₂, R₃, R₉ₐ, and R_{9b} are each independently selected from the group consisting of hydrogen, alkyl, cycloalkyl, and aryl;

R₄ is selected from the group consisting of hydrogen, —OR₂₀, —SR₂₀, —C(O)OR₂₀, and —C(O)R₂₀;

R₅-R₈ and R₁₀-R₁₄ are each independently selected from the group consisting of hydrogen, —OR₂₀, —SR₂₀, —C(O)OR₂₀, —C(O)R₂₀, alkyl, cycloalkyl, and aryl;

R₂₀ is selected from the group consisting of hydrogen, alkyl, cycloalkyl, and aryl; and

n is an integer between 0-10, for example n is 1, 2, 3, 4, 5, 6, 7, 8, 9, or 10.

In some embodiments, R₁ is selected from the group consisting of hydrogen, methyl, ethyl, propyl, n-butyl, and t-butyl. In some of these embodiments, R₁ is hydrogen.

In some embodiments, R₂ and R₃ are each independently selected from the group consisting of hydrogen, methyl, ethyl, propyl, n-butyl, and t-butyl. In some of these embodiments, R₂ and R₃ are hydrogen.

In some embodiments, R₉ₐ and R_{9b} are each independently selected from the group consisting of hydrogen, methyl, ethyl, propyl, n-butyl, and t-butyl. In some of these embodiments, R₉ₐ and R_{9b} are hydrogen.

In some embodiments, R₄ is selected from the group consisting of hydrogen, —OR₂₀, —SR₂₀, —C(O)OR₂₀, and —C(O)R₂₀; wherein R₂₀ is selected from the group consisting of hydrogen, methyl, ethyl, propyl, n-butyl, t-butyl, and phenyl. In some of these embodiments, R₄ is selected from the group consisting of —OH, —OCH₃, and —OPh (phenoxy). In certain of these embodiments, R₄ is —OH.

In some embodiments, R₅-R₈ are each independently selected from the group consisting of hydrogen, methyl, ethyl, propyl, n-butyl, and t-butyl. In some of these embodiments, R₅ and R₈ are hydrogen. In some of these embodiments, R₆ and R₇ are methyl.

In some embodiments, R₁₀ and R₁₄ are each independently selected from the group consisting of hydrogen, methyl, ethyl, propyl, n-butyl, and t-butyl. In some of these embodiments, R₁₀ and R₁₄ are hydrogen.

In some embodiments, R₁₁ and R₁₃ are each independently selected from the group consisting of hydrogen, methyl, ethyl, propyl, n-butyl, and t-butyl. In some of these embodiments, R₁₁ and R₁₃ are t-butyl.

In some embodiments, R₁₂ is selected from the group consisting of hydrogen, —OR₂₀, —SR₂₀, —C(O)OR₂₀, —C(O)R₂₀, methyl, ethyl, propyl, n-butyl, and t-butyl, wherein R₂₀ is selected from the group consisting of hydrogen, methyl, ethyl, propyl, n-butyl, t-butyl, and phenyl. In certain of these embodiments, R₂₀ is hydrogen. In some embodiments, R₁₂ is —OH.

In some embodiments, n is an integer between 1-5, for example, 1, 2, 3, 4, or 5. In some of these embodiments, n is 2, whereas in other embodiments, n is 3.

In another aspect, disclosed herein is the compound designated as CID-2858522 or a pharmaceutically acceptable salt thereof.

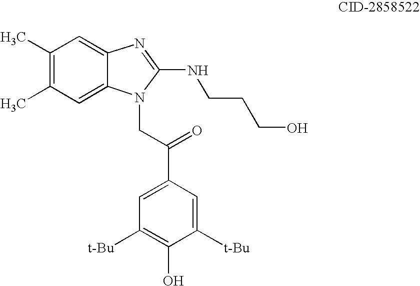

The compounds of the present invention can be synthesized using well-known synthetic organic chemistry techniques. Schemes 1 and 2, below, show synthetic pathways that are used in synthesizing some of the compounds disclosed herein. Additional synthetic procedures are described in the Examples section, below.

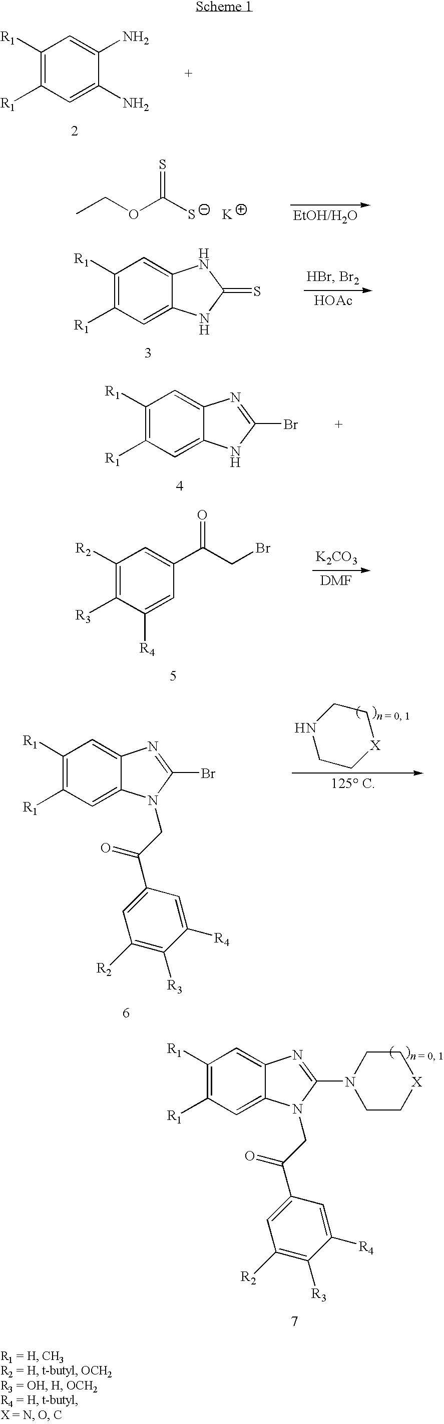

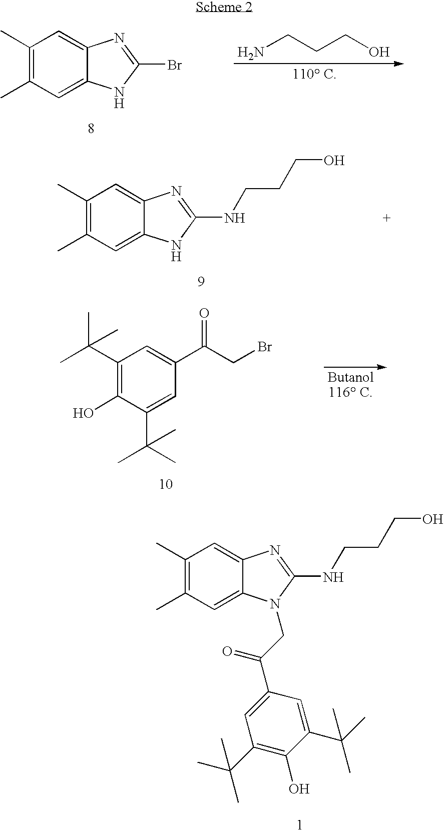

In some embodiments, when the cyclic amine in the final step of Scheme 1 is replaced with an acyclic alkyl amine, two separate products are obtained: an acyclic benzimidazole analog, and a tricyclic benzimidazoimidazole analog. Thus, in some embodiments, the compounds of Formula I can cyclize under acidic conditions, or upon the application of heat, to form compounds of Formula II. An example of such cyclization reaction is shown below in Scheme 3. Certain compounds of Formula I can cyclize to form an analogous compound of Formula II under physiological conditions.

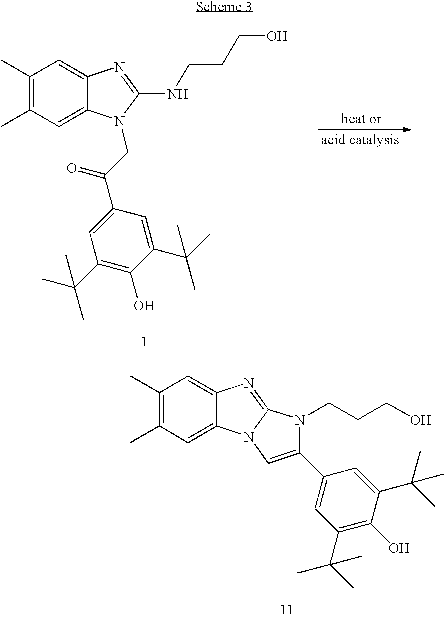

The compounds of the invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

The present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of the invention can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of the compounds of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use alone or with other compounds will vary not only with the particular salt selected but also with the route of administration, the nature of the condition being treated and the age and condition of the patient and will be ultimately at the discretion of the attendant physician or clinician.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound may be conveniently administered in unit dosage form; for example, containing 5 to 1000 mg, conveniently 10 to 750 mg, most conveniently, 50 to 500 mg of active ingredient per unit dosage form.

The active ingredient may be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Methods of Use

In another aspect, disclosed herein is a method of identifying a compound that selectively inhibit antigen receptor-mediated NF-κB activation comprising: (a) providing an aqueous solution comprising a cell transfected with a reporter gene driven by a NF-κB responsive promoter; (b) adding to the solution a test compound; (c) adding to the solution an NF-κB inducing stimulus; and (d) determining whether the test compound reduces the cell response to the stimulus. In some embodiments, the test compound is a compound of Formula I or Formula II, as described herein. In certain of these embodiments, the test compound is CID-2858522. In some embodiments, the reporter gene is a luciferase reporter gene driven by a NF-κB responsive promoter. In other embodiments, the test compound reduces the response to the stimulus by greater than 50 percent.

In another aspect, disclosed herein is a method of selectively inhibiting antigen receptor-mediated NF-κB activation in a cell comprising contacting the cell with a compound of Formula I or Formula II, as described herein. In certain of these embodiments, the compound of Formula I is CID-2858522. In some embodiments, the contacting is in vivo, whereas in other embodiments, the contacting is in vitro.

In another aspect, disclosed herein is a method of selectively inhibiting antigen receptor-mediated NF-κB activation in a subject comprising identifying a subject in need thereof and administering to the subject, or contacting the subject with, a compound of Formula I or Formula II, as described herein. In certain of these embodiments, the compound of Formula I is CID-2858522. In some embodiments, the subject is a mammal. In certain of these embodiments, the subject is a human.

In another aspect, disclosed herein is a method of treating a disease associated with antigen receptor-mediated NF-κB activation in a subject comprising identifying a subject in need thereof and administering to the subject, or contacting the subject with, a compound of Formula I or Formula II, as described herein. In certain of these embodiments, the compound of Formula I is CID-2858522. In some embodiments, the subject is a mammal. In certain of these embodiments, the subject is a human.

### Compound Library Screening

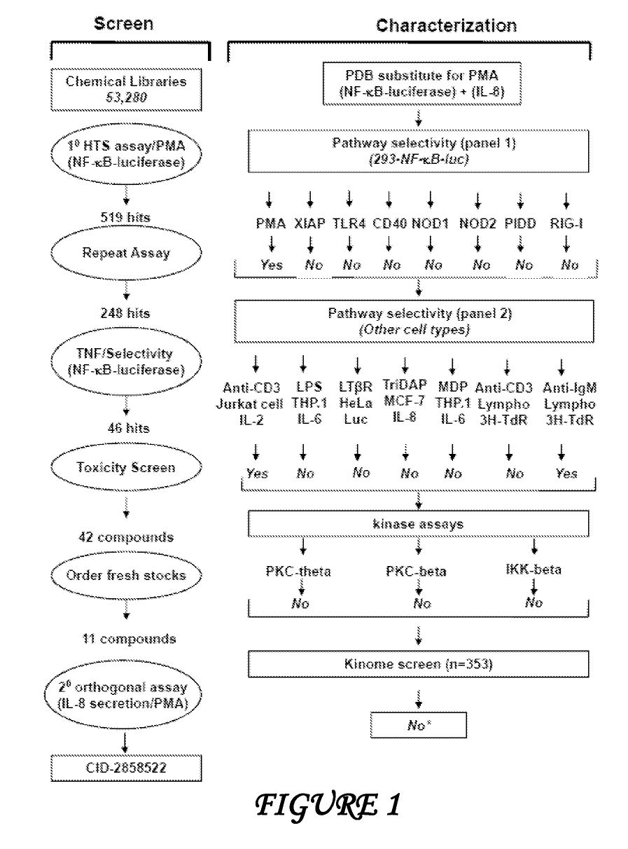

The strategy for compound library screening entailed using phorbol ester (phorbol myristic acetate [PMA]) and Ca²⁺-ionophore Ionomycin to achieve PKC activation, mimicking the initiating events in the antigen receptor pathway. For convenience, we used HEK293 epithelial cells, in which it has previously been shown by siRNA-mediated gene silencing and transfection of dominant-negative mutants that PMA/Ionomycin-induced NF-κB activation is dependent on CARMA1, Bcl-10, and MALT. HEK293 cells were stably transfected with a luciferase reporter gene driven by a NF-κB responsive promoter, and the responsiveness of this integrated promoter to various NF-κB inducing stimuli was confirmed, including PMA/Ionomycin and TNF. Using these cells, 96- and 384-well plate-based high throughput screening (HTS) assays were established, with good assay performance characteristics (Z′>0.5) (PubChem AID=1384). We initially screened 53,280 chemical compounds at an average concentration of 5 μM, of which 519 primary hits were obtained (based on cut-off of 50% inhibition). Of these, 248 confirmed upon repeat testing (FIG. 1).

FIG. 1 shows the results of the primary assay, as well as dose-response experiment. The validated hits with IC₅₀<3 μM were further characterized with counter-screens to assess pathway selectivity. The most potent compound from the NIH library failed to suppress in a secondary assay in which an endogenous NF-κB inducible gene encoding IL-8 was measured. We then screened 53,280 compounds from the Burnham Institute for Medical Research. 248 of 519 compounds were reconfirmed using the same primary screening assay. These compounds were further tested using counter-screening assays to identify pathway-selective inhibitors. Eleven compounds appeared to selectively inhibit NF-κB activated by phorbol ester but not by TNF. From these eleven active compounds, one hit was identified to selectively inhibit PMA/Ionomycin-activated NF-κB pathway with cellular potency of <0.1 μM (IC₅₀) using NF-κB reporter gene assays and also using assays where secretion of NF-κB-induced cytokine IL-8 is measured. The hit also partially inhibits IL-2 production in Jurkat T-cell line but failed to inhibit CD40, CD4, NOD1, NOD2 overexpression induced NF-κB. CID-2858522 did not inhibit IKK and PKC in in vitro kinase assays, indicating the specificity of CID-2858522 in antigen receptor pathway.

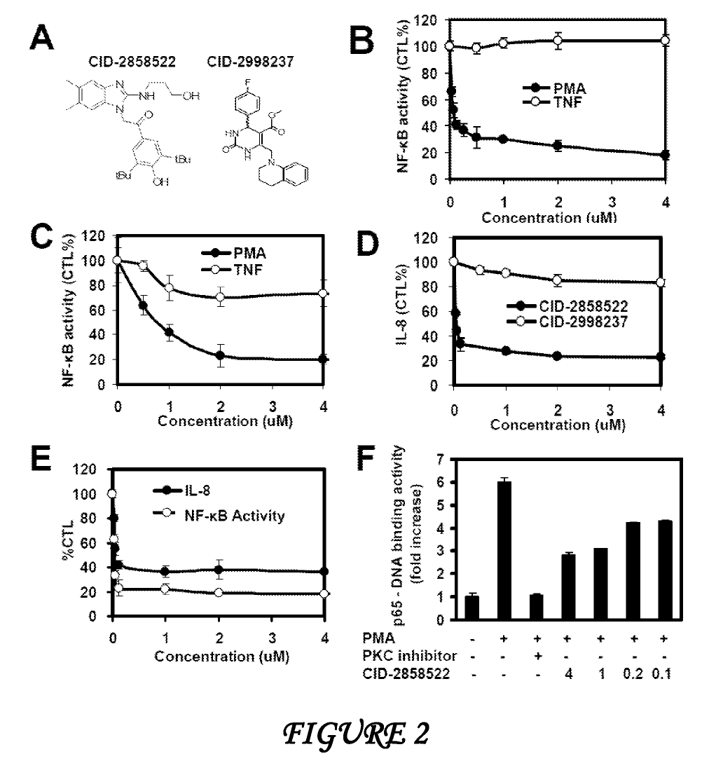

Counter-screening for compounds that inhibit TNFα-induced activation of the reporter gene eliminated 202 compounds and testing for cytotoxicity of the HEK293 reporter cell line discounted two additional compounds leaving 46 candidates. Fresh stocks were ordered of these chemicals, of which eleven showed suppression of PMA-induced NF-κB reporter gene activity. Finally, these eleven candidates were tested in an orthogonal assay in which PMA-induced secretion of Interleukin-8 (IL-8) by the HEK293 reporter cell line was measured, thus examining an endogenous NF-κB target gene, leaving only one candidate compound, CID-2858522. (SID-17450324 or ChemBridge-5653914), a 2-aminobenzimidzole (FIG. 1; FIG. 2A). CID-2858522 also inhibited NF-κB activation induced by another PKC activator, phorbol dibutryate (PDBu), with cellular potency of <0.1 μM (IC50) using NF-κB reporter gene assays and using assays where secretion of NF-κB-induced cytokine IL-8 is measured (FIG. 2E). Attempts to garner additional compounds from a 61,609 library provided by the NIH using the same HEK293-reporter gene cell line in a primary HTS assay formatted for 384 well plates and using a similar followed-up strategy for compound characterization (PubChem AID 586 and AID 465) resulted in no compounds that fulfilled the desired criteria.

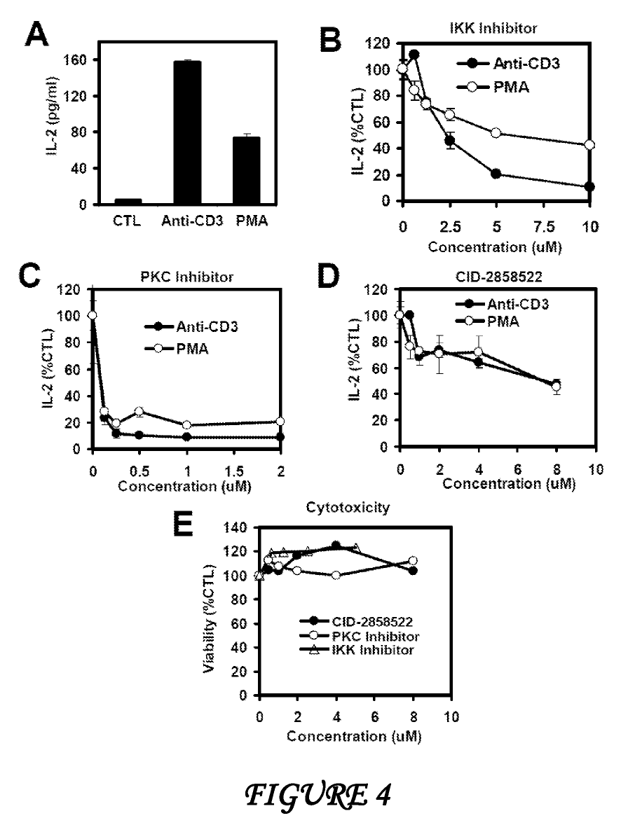

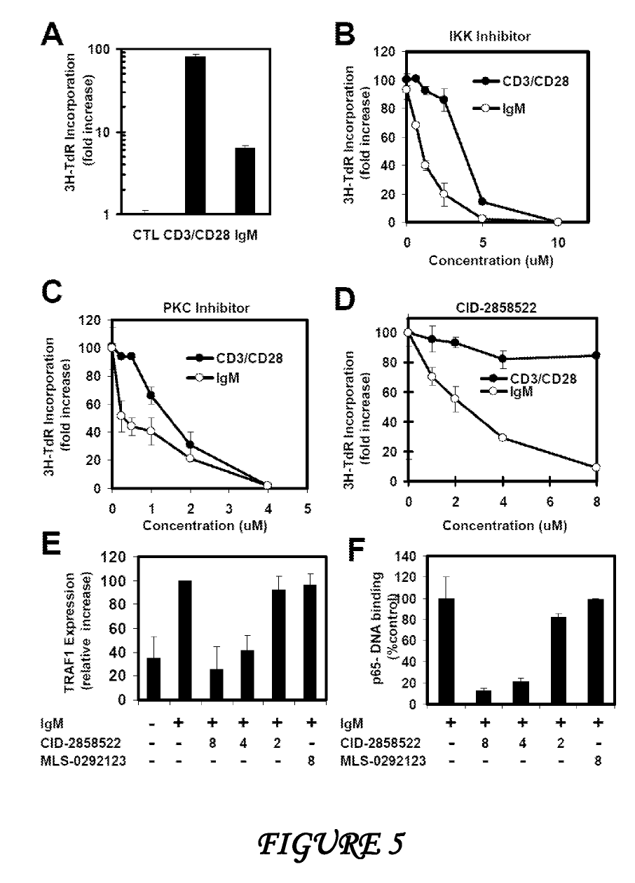

To characterize the specificity of the hit, CID-2858522 was also tested in eight other NF-κB pathways. CID-2858522 did not inhibit the NF-κB activation induced by overexpression of CD40, CD4, NOD1, NOD2, XIAP/TAB, IAP2/MALT1 or induced by either Doxorubicin (an inducer of PIDD, p53-inducible death domain) or Retinoic acid (an inducer of RIG-1), confirming the specificity of CID-2858522 for the antigen receptor pathway. To further confirm the activity and specificity of CID-2858522, we then tested the compound in other cell lines stimulated by various stimuli. CID-2858522 also partially inhibited IL-2 production in Jurkat T-cell line (FIG. 4) and proliferation of mouse B-cell splenocytes (FIG. 5) induced by anti-IgM but failed to inhibit the NF-κB induced by lipopolysaccharide (LPS) (IL-6 secretion measured in THP.1 cell cultures), anti-Lymphotoxin-β receptor (NF-κB luciferase measured in HeLa cells), γ-Tri-DAP (IL-8 secretion was measured in MCF-7 cell cultures) and MDP (IL-6 measured in THP. 1 cell cultures) (data not shown).

Testing >250 analogs of CID-2858522 using the HEK293-NF-κB-luc reporter cell line demonstrated robust structure-activity relations (SAR), in which various moieties within the compound structure were interrogated, resulting in over 200 structurally related analogs that lost all activity or had markedly reduced activity, and approximately ten analogs with comparable activity, but no analogs with clearly superior activity (data to be published elsewhere).

CID-2858522 potently and selectively inhibits phorbol ester-stimulated NF-κB activity. Compound CID-2858522 is a 2-aminobenzimidazole (FIG. 2A). Representative data are provided in FIG. 2, contrasting the activity of CID-2858522 with another compound derived from library screening, CID-2998237 (FIG. 2A) and with a PKC inhibitor, Bisindolylmaleimide I. In the HEK293 cell line used for primary screening, CID-2858522 suppressed NF-κB reporter gene activity in a concentration-dependent manner, with IC₅₀˜70 nM and with maximum inhibition achieved at 0.25-0.5 μM (FIG. 2B). In contrast, this compound did not inhibit TNF-induced NF-κB reporter gene activity at concentrations as high as 4 μM, thus demonstrating selectivity for the NF-κB pathway activated by PMA/Ionomycin (FIG. 2B). Cell viability assays indicated that CID-2858522 was not toxic to HEK293 cells at concentrations ≦8 μM and that it did not inhibit luciferase activity as measured by an in vitro enzymatic assay using purified luciferase (not shown), thus eliminating these trivial explanations for the NF-κB inhibitory activity. Moreover, CID-2858522 also potently inhibited PMA/Ionomycin-induced NF-κB reporter gene activity in transient transfection assays, where the NF-κB-luciferase reporter gene activity was measured from an episomal plasmid (not shown), thus excluding an impact of the chromosomal integration site on measured activity. Similar results were obtained with another “hit” compound CID-2998237, though the compound was less potent at suppressing PMA/lonomycin-induced reporter gene activity and it showed some modest inhibition of TNF-induced NF-κB activity (FIG. 2C).

The orthogonal assay for PMA/Ionomycin-stimulated NF-κB reporter gene activity in the HEK293 engineered cell line used for primary screening proved to be a key differentiator of true-positive versus false-positive compounds, and demonstrates the importance of not relying exclusively on luciferase-based reporter genes. FIG. 2D compares CID-2858522 with a false-positive compound, CID-2998237, showing that CID-2858522 inhibits PMA/Ionomycin-stimulated IL-8 production in a concentration-dependent manner, with IC50<0.1 μM and maximum suppression achieved at 1 μM, whereas CID-2998237 had minimal effect on IL-8 production at concentrations as high as 4 μM. While several compounds derived from library screening demonstrated similar characteristics with respect to suppression of NF-κB reporter gene activity induced by PMA/Ionomycin but not TNF (n=18 of 53,280 total compounds screened), only CID-2858522 suppressed PMA/lonomycin-induced IL-8 secretion.

Similar results for CID-2858522 were obtained when phorbol dibutryate (PDB) was substituted for PMA (FIG. 2E), thus extending the observations to an alternative PKC-activating phorbol ester. The IC₅₀ values for suppression of PDB-induced NF-κB reporter gene activity and PDB-induced IL-8 production by HEK293 cells were 70 nM and 100 nM, respectively.

CID-2858522 also suppressed PMA/Ionomycin-stimulated NF-κB DNA-binding activity (FIG. 2F), as measured by an immunoassay wherein nuclear NF-κB-family proteins are captured on beads displaying oligonucleotides with NF-κB-binding sites and p65 Rel-A is detected using a specific antibody. Suppression was evident at concentrations as low as 0.1 μM and maximal at ˜1 μM. However, CID-2858522 only partially inhibited PMA/lonomycin-induced p65-RelA DNA-binding activity, compared to PKC inhibitor, Bisindolylmaleimide I used here as a control. When compared with more complete suppression of PMA/Ionomycin-driven NF-κB reporter gene activity and IL-8 production, this observation suggests that perhaps PMA/Ionomycin-induced activation of additional members of the NF-κB family besides p65-RelA is inhibited by CID-2858522 in HEK293 cells and that these other NF-κB family members contribute to the total NF-κB reporter gene activity and the IL-8 gene activity measured. CID-2858522 did not block p65-DNA binding activity induced by TNF (data not shown), thus demonstrating pathway selectivity.

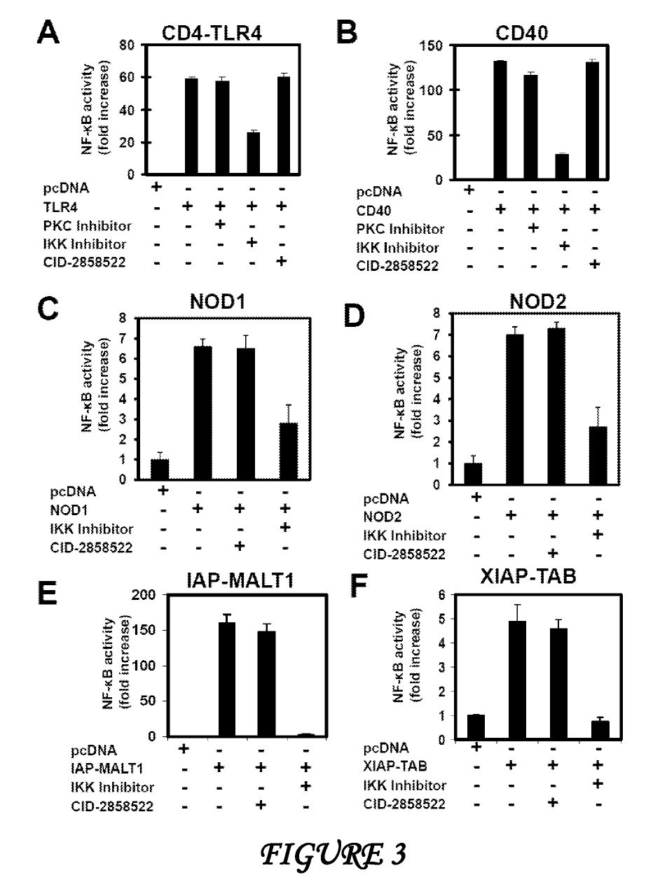

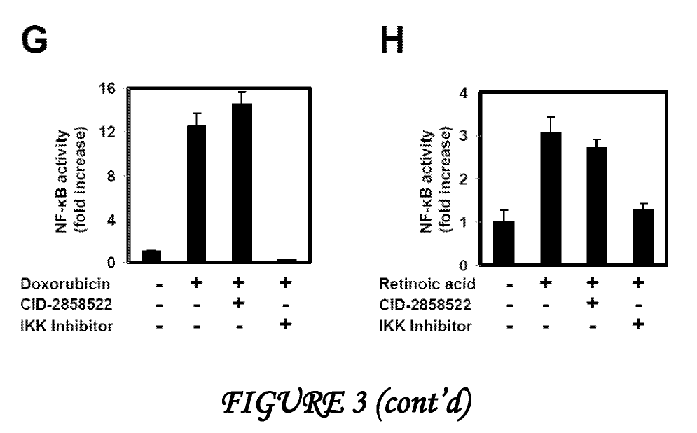

CID-2858522 does not inhibit other NF-κB pathways. Because NF-κB can be activated by at least nine known pathways, we next triggered each of these pathways in HEK293 cells by either stimulation with appropriate cytokines transfection with plasmids, or stimulation with various agents that initiate each NF-κB activation pathway (FIG. 3). The activity of CID-2858522 was compared with an IKK inhibitor, BMS-345541 as a control, relying on the ability of chemical inhibitors of IKKs to block all NF-κB activation pathways. First, we stimulated the TLR-pathway by transfection with a CD4-TLR4 fusion protein, in which the extracellular domain of CD4 is fused with the transmembrane and cytosolic domain of TLR4, and whereby anti-CD4 antibody (rather than the natural ligand, lipopolysaccharide [LPS]) is used to activate TLR4. TLR4 induced robust NF-κB reporter gene activity (>50 fold increase), which was suppressed by IKK inhibitor BMS-345541, but not by CID-2858522 and not by PKC inhibitor, Bisindolylmaleimide I. Second, the “alternative” NF-κB pathway was stimulated by over-expressing CD40 in HEK293 cells. CD40-induced NF-κB reporter gene activity was potently suppressed by the IKK inhibitor but not by CID-2858522 or by the PKC inhibitor. Third, we stimulated the NLR-dependent NF-κB pathway by over-expressing NOD1 (NLRC1) or NOD2 (NLRC2) in the HEK293-NFκB-luc cells. NOD1 and NOD2 induced 6-7 fold increases in NF-κB-luciferase reporter gene activity, which were inhibited by the IKK inhibitor but not by CID-2858522. Fourth, IAP-initiated pathways for NF-κB activation were induced by transfecting 293-NF-κB-luciferase cells with plasmids encoding either cIAP2/MALT oncoprotein or XIAP plus TAB. While an IKK inhibitor effectively suppressed these IAP-driven pathways, CID-288522 did not. Fifth, the DNA-damage-inducible pathway for NF-κB activation was triggered by stimulating HEK293-NF-κB-luc cells with doxorubicin, which induced 12-fold increase in NF-κB activity in these cells. Again, the IKK inhibitor suppressed NF-κB activity but not CID-2858522. Finally, the retinoic acid (RA)-inducible pathway involving RIG-I was induced by treating cells with all-trans-retinoic acid. RA induced a modest 3-fold increase in NF-κB activity in HEK293 cells, which was significantly suppressed by the IKK inhibitor but not by CID-2858522. Thus, when taken together with the data showing that the “classical” NF-κB pathway activated by TNF is not inhibited by CID-2858522 (FIG. 2), these data demonstrate that our compound uniquely suppresses the NF-κB pathway initiated by PKC activators.

CID-2858522 partially inhibits TCR-stimulated IL-2 production by Jurkat T cells. In T cells, the antigen receptor stimulates several signal transduction pathways that converge on the IL-2 gene promoter, including NF-κB, NF-AT, and AP-1. For evaluating the effects of CID-2858522 on TCR-initiated, NF-κB-driven events in lymphocytes, we employed Jurkat T-leukemia cells, which have been utilized extensively as model for studying TCR-signaling leading to IL-2 gene expression. For these experiments, Jurkat cells were stimulated with either anti-CD3 (to activate the TCR complex) and anti-CD28 (co-stimulator) or with PMA/Ionomycin, in the presence or absence of CID-2858522, IKK inhibitor, or PKC inhibitor, then IL-2 production was measured 24 hrs later in culture supernatants. Both anti-CD3/CD28 and PMA/ionomycin stimulated marked increases in IL-2 production by Jurkat T-cells, with CD3/CD28 more potent than PMA/Ionomycin (FIG. 4A). The IKK inhibitor partially suppressed PMA/Ionomycin-induced IL-2 production, and essentially completely (90% suppression) inhibited anti-CD3/CD28-induced IL-2 production by concentrations ≦10 μM (FIG. 4B). The PKC inhibitor suppressed IL-2 production by 80-90% in Jurkat cell stimulated with either CD3/CD28 or PMA/Ionomycin at concentrations <0.5 μM (FIG. 4C). In contrast, CID-2858522 suppressed IL-2 production by CD3/CD28- and PMA/Ionomycin-stimulated Jurkat cells by approximately half (IC₅₀) at concentrations <10 μM (FIG. 4D). The suppression of IL-2 production by Jurkat cells by CID-2858522, IKK inhibitor, or PKC inhibitor was not due to cytotoxicity (FIG. 4E).

In contrast to its suppression of IL-2 production by CD3/CD28-stimulated Jurkat T-cells, CID-2858522 did not suppress IL-6 production by THP.1 monocytes stimulated with TLR4 agonist LPS, IL-8 production stimulated by NOD1 agonist γ-TriDAP in MCF7 breast cancer cells, or NF-κB luciferase activity induced by Anti-Lymphotoxin-β in HeLa cells (as summarized in FIG. 1), all of which involve other NF-κB activation pathways. Thus, CID-2858522 also demonstrated pathway selectivity when triggering endogenous components of several NF-κB activation pathways rather than relying on gene transfection.

CID-2858522 inhibits mouse primary B cell proliferation induced by anti-IgM. NF-κB plays roles in antigen receptor-driven lymphocyte proliferation. We therefore tested the effect of CID-2858522 on mouse lymphocyte (both B-cells and T-cells) proliferation induced by anti-CD3/CD28 or anti-IgM antibodies, measuring ³H-Thymidine incorporation. Anti-CD3/CD28 and anti-IgM significantly induced 80-fold and 8-fold increases, respectively, in DNA synthesis in cultures of murine lymphocytes (FIG. 5A). The IKK and PKC inhibitors suppressed lymphocyte proliferation in a concentration-dependent manner, inhibiting B-cells (anti-IgM) (IC₅₀˜2 μM of IKK inhibitor; 0.2 μM for PKC inhibitor) more potently than T-cells (anti-CD3/CD28) (IC₅₀˜4 μM for IKK inhibitor; 11.5 μM for PKC inhibitor) (FIGS. 5B, C). In contrast, CID-2858288 inhibited anti-IgM-induced lymphocyte proliferation in a concentration-dependent manner, with IC₅₀ 2 μM, while having minimal effect on anti-CD3/CD28, suggesting that the NF-κB inhibitory mechanism of CID-2858288 is more prominent in B-cell versus T-cells. However, because CD3/CD28 stimulates stronger proliferation responses than anti-IgM, we cannot exclude a quantitative rather than qualitative explanation for this observation.

To further evaluate the effect of CID-2858522 on antigen receptor signaling in lymphocytes, we employed B-cells from patients with Chronic Lymphocyte Leukemia (CLL), where >90% of the peripheral blood lymphocytes are neoplastic B-cells. Stimulation with biotinylated anti-IgM (crosslinked using Streptavidin) resulted in expression of TRAF1 (FIG. 5E), an endogenous target of NF-κB. Adding CID-285252 to cultures of anti-IgM-stimulated CLL B-cells inhibited TRAF1 induction, in 3 of 3 cases measured at 24 hr after stimulation (FIG. 5E). Levels of Actin and TRAF6, which are not regulated by NF-κB, did not show any change, thus showing selectivity and confirming equivalent protein loading. As a control, CLL B-cells were also treated by a structurally related but inactive 2-aminobenzimidazole analog, MLS-0292123, which does not inhibit PMA/Ionomycin-induced NF-κB luciferase activation or IL-8 production in HEK293 cells, showing that MLS-0292123 did not inhibit TRAF1 expression (FIGS. 5E, 5F). As a positive control, CLL B-cells were also treated with a PKC inhibitor, Bisindolylmaleimide I, which also inhibited TRAF1 expression. The effects of CID-2858522 on anti-IgM-stimulated TRAF1 expression were not due to cytotoxicity during the time-frame analyzed, as confirmed by measuring ATP levels. In addition to the indirect evidence of NF-κB activation using TRAF1 expression, CID-2858522 also showed direct suppression on p65-DNA binding activity in human CLL B-cells induced by anti-IgM, while its inactive analog, MLS-0292123 did not (FIG. 5E). Thus, CID-2858522 inhibits the B-cell antigen receptor-stimulated NF-κB activation.

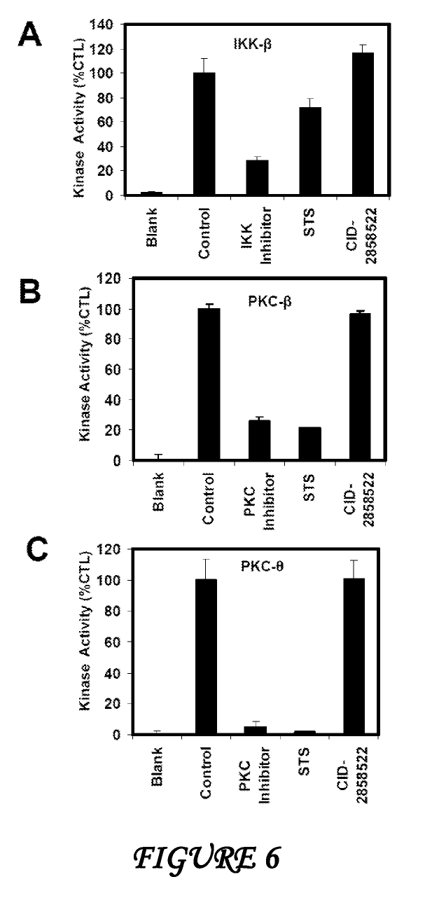

CID-2858522 is not a potent protein kinase inhibitor. Protein kinases play critical roles in NF-κB activation. PKCs are proximal kinases in the NF-κB pathways activated by PMA/Ionomycin and by T- and B-cell antigen receptors, while the IKKs are distal kinases operating in the terminal segments of these and other NF-κB activation pathways. We therefore tested whether CID-2858522 inhibits members of these kinase families by in vitro kinase assays. For these experiments, we tested PKC-beta and PKC-theta (the PKC family members implicated in TCR/BCR signaling), and IKK-beta (a component of the IKK complex) (FIG. 6). At concentrations up to 8 μM, CID-2858522 failed to suppress these kinases, while known PKC and IKK inhibitors and the broad-spectrum kinase inhibitor staurosporin (STS) demonstrated potent inhibition. Thus, CID-2858522 does not directly inhibit PKC-beta, PKC-theta, or IKK-beta. In addition to assessing these three kinases by conventional in vitro kinase assays, a kinome screen was performed using a high throughput screening method, KINOMEscan™, which is an active-site dependent competition binding assay in which human kinases of interest are fused to a proprietary tag (Ambit). The amount of kinase bound to an immobilized, active-site directed ligand is measured in the presence and absence of the test compound. Of 353 protein kinases surveyed, CID-2858522 suppressed by >50% at 10 μM only 3 protein kinases: Raf (57% inhibition), TLK1 (70% inhibition), and JAK2 (53% inhibition), none of which are clearly implicated in NF-κB regulation. Thus, CID-2858522 inhibits none of the protein kinases previously implicated in regulating NF-κB.

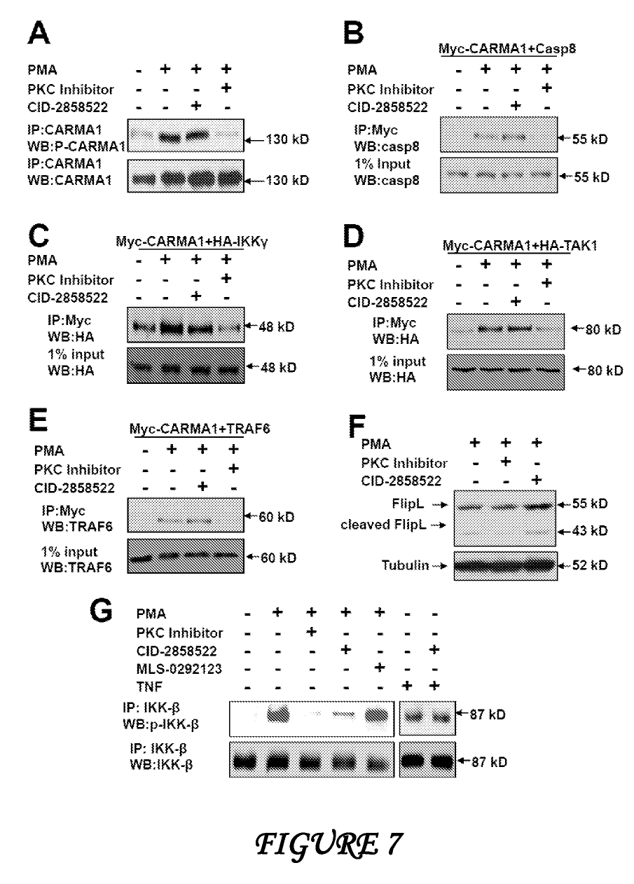

Mapping the site of action of CID-2858522 in the antigen receptor-activated NF-κB pathway. Based on these kinase screens, we deduced that CID-2858522 operates somewhere between PKCs and IKK to inhibit the NF-κB pathway involved in antigen receptor signaling, which is known to include CARMA-family proteins, Bcl-10, MALT, TRAF6 (which binds Ubc13 to induce lysine 63-linked polyubiquitination of IKKγ/NEMO), IKKγ, and Caspase-8. To characterize the effects of CID-2858522 on these possible targets of the antigen receptor pathway for NF-κB activation, we first evaluated PMA-induced phosphorylation of Carmal, by phospho-specific antibody immunoblotting, finding no effect of CID-2858522 on this molecular event that initiates formation of the CBM complex (FIG. 7A). Next, we performed co-immunoprecipitation (co-IP) experiments, assessing the interactions of Bcl-10, MALT, TRAF6, IKKγ, and Caspase-8 with either CARMA1 or CARMA3 in transfected HEK293 cells, before and after stimulation with PMA. PMA induced or increased association of CARMA1 or CARMA3 with each of these proteins, which was inhibited by a PKC inhibitor, Bisindolylmaleimide I, but not by CID-2858522 (FIG. 7B-E). CID-2858522 did not disrupt the formation of CARMA/MALT1/Bcl-10 (CMB) complex induced by PMA/Ionomycin in either cells or lysate.

Caspase-8 plays an essential role in antigen receptor-mediated NF-κB activation. It was recently reported that MALT1 interacts with Caspase-8 and activates this protease upon antigen receptor activation. We confirmed that, in HEK293 cells too, caspase-8 activation was required for the NF-κB activation as z-ITED-fmk, a specific caspase-8 inhibitor, or caspase-8 siRNA can significantly inhibit NF-κB luciferase activation induced by PMA/Ion. We then examined if CID-2858522 affected the pathway at this point. PMA induced significant MALT1-caspase-8 interaction in HEK293 cells over-expressing Flag-MALT1 and Caspase-8. The interaction was inhibited by a PKC inhibitor but not by CID-2858522. The caspase-8 p43/41 processing intermediate was generated in HEK293 cells after PMA/Ion treatment. However, it was inhibited by a PKC inhibitor but not by CID-2858522 (data not shown). We then assessed the effects of CID-2858522 on PMA-induced proteolytic processing of c-FLIP, a Caspase-8-mediated event recently shown to be required for antigen receptor mediated NF-κB activation. Immunoblot analysis of lysates from HEK293 cells following stimulation with PMA/Ionomycin showed c-FLIP processing (FIG. 7F), which was completely inhibited by the PKC inhibitor but not affected by CID-2858522. Thus, CID-2858522 failed to inhibit Caspase-8 activation and c-FLIP processing.

Finally, we examined IKK-β phosphorylation. Phosphorylation of IKK-β was induced by PMA/Ionomycin in HEK293 cells and was significantly inhibited by CID-2858522, but not by its inactive analog, MLS-0292123 (FIG. 7G). In contrast, CID-2858522 failed to inhibit TNF-α-induced IKK-β phosphorylation, indicating pathway selectivity. We concluded from these studies that CID-2858522 inhibits PMA/Ionomycin-induced NF-κB at a point downstream of CBM complex formation, caspase-8 activation and c-FLIP processing, but upstream of IKK-β phosphorylation.

Chemical inhibitors of NF-κB have been widely sought for potential use as therapeutics for autoimmunity, inflammation, and cancer. However, the most pharmaceutically tractable of the NF-κB-activating targets, the IKKs, represent a shared component of all known NF-κB activation pathways and thus lack selectivity. In this regard, NF-κB activity is required for innate immunity and host-defense against microorganisms and various viral and bacterial pathogens. In addition to impaired host defense, broad-spectrum suppression of NF-κB pathways may reduce basal NF-κB activity and interfere with the function of NF-κB as a survival factor, leading to potentially toxic side effects. For example, IKK-β knockout mice die at mid-gestation from uncontrolled liver apoptosis. Moreover, from the standpoint of generating research tool compounds for basic research, it would be useful to have pathway-selective inhibitors that reveal in what cellular contexts a particular pathway is important for specific cellular responses.

Using a chemical biology strategy, we devised chemical library screens for inhibitors that selectively inhibit the NF-κB activation pathway induced by PKCs and antigen receptors. This pathway is uniquely involved in acquired immunity (rather than innate immunity), and has been linked to numerous autoimmune diseases and some types of lymphomas and lymphocytic leukemia. Also, because PKC hyperactivity has been associated with some solid tumors the pathway interrogated here may also be relevant to a variety of malignancies. The NF-κB activation pathway linked to PKCs and antigen receptors is known to involve proteins unique to this pathway among the nine known NF-κB activation pathways—namely, CARMA (Bimp)-family proteins, Bcl-10, and MALT. Upon phosphorylation of CARMA1 by PKC in the context of antigen receptor signaling, these proteins form a complex, which recruits TRAF6, an E3 ligase that binds Ubc13, resulting in lysine 63-linked poly-ubiquitination of IKKγ/NEMO, resulting in IKK activation. Caspase-8 is also recruited, resulting in proteolytic processing of c-FLIP, an event required for antigen receptor-induced activation of NF-κB. The components of this complex required for IKK activation may not be completely known and an active complex has not been reconstituted in vitro using purified components, thus making biochemical screens difficult. For this reason, a cell-based strategy for chemical library screening was the only practical option.

Using HEK293 cells containing an NF-κB-driven reporter gene stimulated by PMA/lonomycin, followed by an orthogonal screen in which we measured levels of the protein product of an endogenous NF-κB target gene (e.g. IL-8) secreted by these same cells, we screened 114,889 compounds, resulting in only 1 compound with the desired properties, CID-2858522. This 2-aminobenzimidazole compound potently inhibits NF-κB reporter gene activity and IL-8 production induced by PKC activators in HEK293 cells, with IC₅₀<0.1 μM, while failing to inhibit NF-κB reporter gene activation by agonists of the other eight NF-κB activation pathways (FIG. 1). CID-2858522 also partially suppressed CD3/CD28- and PMA/Ionomycin-stimulated IL-2 production by Jurkat T-cells and proliferation of anti-IgM-stimulated primary murine lymphocytes (FIGS. 4 and 5), phenotypes expected for a selective antagonist of the NF-κB activation pathway activated by antigen receptors.

The observation that IL-2 production by CD3/CD28- and PMA/Ionomycin-stimulated Jurkat T cells was only partially suppressed by CID-2858522 may be consistent with knowledge that NF-κB is only one of several transcriptional regulators of the IL-2 gene promoter, which includes NF-κB, NFAT, and AP-1. Similarly, given that a variety of NF-κB-activating cytokines are elaborated upon stimulation of cultured lymphocytes with antibodies cross-linking CD3 (TCR) or surface IgM (BCR), it is perhaps not surprising that CID-2858522 only partially suppressed proliferation of anti-IgM-stimulated primary B-cells and had minimal effect on anti-CD3/CD28-stimulated T-cell proliferation. In contrast, an IKK inhibitor essentially completely suppressed lymphocyte proliferation at concentrations of 5 μM, consistent with its ability to neutralize all known NF-κB activation pathways. CID-2858522 also inhibited anti-IgM-stimulated expression of the endogenous NF-κB target gene, TRAF1, in CLL B-cells. In this regard, the TRAF1 gene promoter contains four NF-κB target sites and a TATA-box, but essentially no other recognizable transcriptional elements, thus making it a good surrogate marker of NF-κB activity in primary cells. Although the mechanisms involved in antigen receptor-mediated NF-κB activation (upstream of PKC activation) in T cells and B cells are distinct, the downstream events following PKC activation share great similarity. Knockout mice models showed that CARMA1, Bcl-10 and MALT1 are required for antigen receptor-induced NF-κB activation and proliferation of both T cells and B cells. However, CARMA1 mutant mice exhibited normal T but impaired B cell development and MALT1 deficiency has only mild effects on B cell activation MALT1, indicating that while the signal transduction apparatus by which antigen receptors stimulate NF-κB downstream of PKC activation in T cells and B cells share great similarity, they may not be identical. In this regard, it is also possible that antigen receptors and other upstream activators of PKCs induce NF-κB activation by more than one pathway, with CID-2858522 inhibiting only one of them. For example, we have observed that CID-2858522 inhibits NF-κB activity induced by PMA/Ionomycin in HEK293 but not HEK293T cells, the latter expressing SV40 virus Large T antigen. Thus, the pathways through which PKCs induce NF-κB activity are cell-context dependent, with our compound showing cell-type-dependence. Comparisons of HEK293 and HEK293T cells by transcriptional profiling, phosphoproteomics, or other methods may provide insights into the molecular basis for this cell-type dependence. It will also be interesting to explore whether various lymphocyte subsets differ in their reliance on the NF-κB pathway components that CID-2858522 targets, analogous to HEK293 versus HEK293T cells. These cell-type-specific attributes make CID-2858522 an interesting research tool compound for distinguishing the roles of various NF-κB pathways in biological contexts, a characteristic that may or may not prove to be exploitable from a therapeutic standpoint. The differences in HEK293 vs HEK293T cell sensitivity to CID-2858522 also illustrate the impact of cell line bias in chemical biology experiments. Had HEK293T cells been employed instead of HEK293 cells, CID-2858522 would not have been identified.

The mechanism by which CID-2858522 suppresses PKC-induced NF-κB activity remains to be determined. We mapped the site of action of this compound downstream of PKCs and upstream of IKK-β. PKCs induce phosphorylation of CARMA1, an event that was not inhibited by CID-2858522. This compound also did not inhibit PMA-induced recruitment of Bcl-10, MALT, TRAF6, Caspase-8, or IKKγ to CARMA1/CARMA3, nor did it inhibit caspase-8 activation or FLIP proteolytic processing. CARMA family proteins include 3 members in mammals, which all contain a N-terminal CARD domain followed by a coiled-coil domain, a PDZ domain, a SH3 domain, and a C-terminal guanylate kinase-like (GUK) domain. CARMA1, predominantly expressed in spleen, thymus, and peripheral blood leukocyte (PBL), has been implicated definitively in antigen receptor signaling. In contrast, CARMA3 is expressed in broad range of tissues but not in spleen, thymus, or PBL and CARMA2 is expressed only in placenta. Suppression of selected members of the CARMA family could provide another plausible explanation for partial inhibition by CID-2858522 of events such as IL-2 production by CD3/CD28- or PMA/Ionomycin-stimulated Jurkat cells and proliferation of primary cultured lymphocytes.

In summary, using a chemical biology approach, we have identified a selective chemical inhibitor of the PKC-initiated NF-κB activation pathway utilized by antigen receptors. This compound and its active analogs provide novel research tools for elucidating the role of this NF-κB pathway in cellular responses, while having the potential to reveal new paths forward for the development of therapeutically useful, pathway-selective NF-κB inhibitors.

The invention will be further described by the following non-limiting examples.

### Example 1

### Chemistry

2-Mercapto-5,6-dimethylbenzimidazole. A mixture of 10 g of 4,5-dimethylphenylenediamine, 16 g of potassium ethyl xanthate, 100 mL ethanol and 14 mL of water were added to a 500 mL Erlenmeyer flask and heated to reflux. After 3 h, 3.4 g of charcoal (Norit A) was added and refluxed for an additional 10 min. The Norit was filtered and the filtrate was heated to 60-70° C. To the warm solution was added 100 mL of warm tap water and 8 mL of acetic acid in 16 mL of water with good stirring. Upon the addition of the acetic acid solution, the mixture became a foamy solid. The mixture was placed in a 4° C. refrigerator for 3 h. The solid was filtered and dried over P₂O₅ to give 8.1 g (62%) of a tan solid. The compound was used without further purification.

2-Bromo-5,6-dimethylbenzimidazole (8). To a cooled solution of 40 mL of acetic acid and 4.2 mL of 48% aqueous HBr was added 5 g 1. To this slurry was added 5.2 mL of bromine dropwise slowly over 10 min. The reaction turned orange and became unstirrable after half of the bromine was added, manual or mechanical agitation was needed to break up solids. After the addition of the bromine, 80 mL of acetic acid was added and the mixture was stirred at room temperature. After 4.5 h, the mixture was diluted with 90 mL of water and cooled to 0° C. The pH of the mixture was adjusted to 4 by the addition of solid NaOH. Upon basification a solid precipitated out of solution. It was filtered and dried overnight to give 2 g (32%) of product as a light orange solid. ¹H NMR (300 MHz, DMSO-d6) δ 2.25 (s, 6H), 7.20 (s, 2H). MS (ESI) calculated C₉H₉BrN₂ m/z=223.99, 225.99 found m/z=225.24, 227.25 [M+H].

General Procedure for Bromination of Acetophenone (5). To a flask containing 6 mL of CH₂Cl₂ was added the appropriate acetophenone (3.1 mmol). To this was added a solution of Br₂ (4.03 mmol) in 12 mL of CH₂Cl₂ under Argon. The reaction was stirred overnight and diluted with 15 mL of CH₂Cl₂. The organic reaction was washed twice with saturated sodium bicarbonate. The organic layer was dried over sodium sulfate and concentrated in vacuo. The crude product was purified using flash chromatography using the appropriate solvent system.

1-(Benzo[d][1,3]dioxol-6-yl)-2-bromoethanone. Isolated 163 mg of a white solid using EtOAc/Hexane 1:3. (TLC EtOAc/Cyclohexane 1:9 R_{f}=0.41). (¹H 300 MHz CDCl₃) δ4.37 (s, 2H), 66.07 (s, 2H), 66.88 (d, J=8.2, 1H), 67.45 (q, J=2.0, 1H), 67.59 (d, J=8.4, 1.7 1H) MS (ESI) (Neg. ion) calcd for C₉H₇BrO₃ [M−H] 242.06,

General Procedure for the Alkylation of Bromobenzimidazole. To a flask was added the appropriate bromobenzimidazole (0.6 mmol), brominated acetophenone (0.66 mmol) and K₂CO₃ (1.3 mmol) in 1.2 mL DMF. The solution was stirred at room temperature overnight. The reaction mixture was poured into 10% Citric Acid and extracted 3× with EtOAc. The organic layers were combined, dried over Na₂SO₄ and concentrated in vacuo. The crude material was purified via column chromatography with the appropriate solvent system.

General Procedure for the synthesis of the benzimidazole library (7). To a 1-dram vial was added the appropriate alkylated bromobenzimidazole 6 (0.01 mmol) and the cyclic amines (0.1 mmol) and heated to 110° C. After 4 h, the reactions were cooled to room temperature and the products were isolated. The reaction mixture was dissolved into 0.5 mL EtOAc and washed with 2 N HCl (0.5 mL). The acid was neutralized with saturated sodium bicarbonate extracted 2× EtOAc (0.5 mL. The organic layers were combined, dried over sodium sulfate and concentrated to dryness. The isolated material was used without further purification.

3-(5,6-Dimethyl-1H-benzo[d]imidazol-2-ylamino)propan-1-ol (9). 50 mg of 8 and 100 μL 3-aminopropanol were added to a 1-dram vial and heated to 125° C. until the reaction was complete based on TLC analysis. The reaction was cooled to room temperature, extracted with saturated sodium bicarbonate/ethyl acetate. The organic layer was dried over sodium sulfate and concentrated to give a brown residue. The crude product was purified via preparative TLC using 10% MeOH/EtOAc. A band isolated with an R_{f}=0.25 gave 37.3 mg (76%) of a light brown residue. MS (ESI) calculated C₁₂H₁₇N₃O m/z=219.14 found m/z=220.56 [M+H].

General Procedure for the Synthesis of the Target Benzimidazoles. To a 1-dram vial, 19 mg of the appropriate alkylaminobenzimidazole and 32 mg of the appropriate bromoacetophenone 5 were added to 0.5 mL butanol. The reaction was heated to 115° C. until the reaction was complete based on TLC analysis. The butanol was removed and the crude mixture was extracted with saturated sodium bicarbonate/ethyl acetate. The organic layer was dried over sodium sulfate and concentrated to dryness. The crude mixture was purified via preparative TLC using 5% MeOH/EtOAc.

2-(2-(3-Hydroxypropylamino)-5,6-dimethyl-1H-benzo[d]imidazol-1-yl)-1-(3,5-di-tert-butyl-4-hydroxyphenyl)ethanone (1). R_{f}=0.3. ¹H NMR (CDCl₃, 300 MHz) δ 0.87-1.00 (m, 2H), 1.49 (s, 18H), 2.22 (s, 3H), 2.26 (s, 3H), 3.52 (t, J=5.6, 2H), 3.61 (t, J=5.5, 2H), 5.60 (s, 2H), 6.87 (s, 1H), 7.05 (s, 1H), 7.97 (s, 2H). MS (ESI) calculated C₂₈H₃₉N₃O₃ m/z=465.30, found m/z=466.82 [M+H].

1-(3,5-di-tert-butyl-4-methoxyphenyl)-2-(2-(3-hydroxypropylamino)-5,6-dimethyl-1H-benzo[d]imidazol-1-yl)ethanone. R_{f}=0.4. MS (ESI) calculated C₂₉H₄₁N₃O₃ m/z=479.31, found m/z=480.53 [M+H].

2-(2-(3-methoxypropylamino)-5,6-dimethyl-1H-benzo[d]imidazol-1-yl)-1-(3,5-di-tert-butyl-4-hydroxyphenyl)ethanone (33). R_{f}=0.4. ¹H NMR (CDCl₃, 300 MHz) δ 1.49 (s, 18H), 1.89-1.97 (m, 2H), 2.28 (s, 3H), 2.29 (s, 3H), 3.25 (s, 3H), 3.52 (t, J=5.6, 2H), 3.60 (t, J=5.5, 2H), 5.16 (s, 2H), 6.79 (s, 1H), 7.27 (s, 1H), 7.91 (s, 2H). MS (ESI) calculated C₂₉H₄₁N₃O₃ m/z=479.31, found m/z=480.23 [M+H].

2-(2-(3-methoxypropylamino)-5,6-dimethyl-1H-benzo[d]imidazol-1-yl)-1-(3,5-di-tert-butyl-4-methoxyphenyl)ethanone (34). R_{f}=0.6. ¹H NMR (CDCl₃, 300 MHz) δ 1.44 (s, 18H), 1.89-1.97 (m, 2H), 2.28 (s, 3H), 2.29 (s, 3H), 3.25 (s, 3H), 3.52 (t, J=5.6, 2H), 3.60 (t, J=5.5, 2H), 3.72 (s, 3H), 5.18 (s, 2H), 6.79 (s, 1H), 7.27 (s, 1H), 7.95 (s, 2H). MS (ESI) calculated C₃₀H₄₃N₃O₃ m/z=493.33, found m/z=494.13 [M+H].

1-(3,5-di-tert-butyl-4-hydroxyphenyl)-2-(2-(butylamino)-5,6-dimethyl-1H-benzo[d]imidazol-1-yl)ethanone (40). R_{f}=0.8. ¹H NMR (CDCl₃, 300 MHz) δ 0.90-0.95 (m, 3H), 1.47 (s, 18H), 1.57-1.68 (m, 4H), 3.44-3.50 (m, 2H), 3.61 (t, J=5.5, 2H), 5.14 (s, 2H), 6.82 (s, 1H), 7.28 (s, 1H), 7.92 (s, 2H). MS (ESI) calculated C₂₉H₄₁N₃O₂ m/z=463.32, found m/z=464.35 [M+H].

### Example 2

### Biochemical/Biological Assays

### Materials and Methods

Reagents. Phorbol myristic acetate (PMA), Ionomycin, muramyl dipeptide (MDP), Retinoic Acid (RA), Doxorubicin and γ-Tri-DAP were from Sigma-Aldrich (St. Louis, Mo.), phorbol dibutryate (PDBu), PKC inhibitor (Bisindolylmaleimide I), and IKK inhibitor (BMS-345541) were from Calbiochem (Gibbstown, N.J.). Anti-mouse-CD3, anti-mouse-CD28, anti-mouse-IgM were obtained from Biomeda (Foster City, Calif.). Anti-human CD3, anti-human CD28 and anti-mouse-IgG antibody were from R&D System (Minneapolis, Minn.). Anti-human TRAF6 antibody has been described. Plasmids encoding HA-IKK-γ, XIAP, HA-TAK1, TAB1, CD4-TLR4CD40, NOD1, NOD2, cIAP1/MALT, Caspase-8 and Caspase-8 (C360S) and TRAF6 have been previously described. Myc-CARMA1 and CARMA3 were gifts from Dr Xin Lin (University of Texas, M. D. Anderson Cancer Center).

Cell engineering. HEK293 cells were co-transfected with pUC13-4xNFκB-Luc and p-TK-puromycin-resistance plasmids. Stable clones were selected by culture in Dulbecco's Modified Eagle's Media (InVitrogen) supplemented with 10% heat-inactivated fetal bovine serum (FBS) (Hyclone), 1% v/v penicillin-streptomycin (InVitrogen) containing 1 μg/mL puromycin. Individual clones were tested for responsiveness to PMA/Ionomycin- and to TNF-induced NF-κB reporter gene activity, and a clone was selected for HTS.

Compounds. Chemical libraries were screened using the cellular NF-κB luciferase reporter assay, including a ChemBridge library (San Diego, Calif.) having 50,000 compounds, Microsource Spectrum collection (Groton, Conn.) having 2,000 compounds, the LOPAC library (Sigma) having 1,280 compounds, and NIH library having 61,609 compounds.

HTS. NF-κB-luciferase expressing HEK293 cells were seeded at 105 per well in white 96 well plates (Greiner Bio-One) in 90 μl of DMEM and incubated overnight. 10 μl of compound-containing solution was added to each well (final 1.5 μg/mL in 1% DMSO) using a liquid handler (Biomek™ FX; Beckman Coulter). After 2 h incubation, cells were stimulated using 11 μl of a PMA (final 100 ng/mL; Calbiochem) and ionomycin (final 50 ng/mL; Calbiochem) in DMEM. Cell plates were incubated for 16 h before media was removed and 40 μl of 0.5× passive lysis buffer (Promega Corp.) was added to cell plates. Plates were allowed to stand at room temperature for at least 15 minutes before adding 40 μl of 0.125× luciferin substrate (Promega Corp.) to each well. Plates were analyzed within 30 seconds with a Criterion Analyst™ using the luminescence method (0.1 second read/well).

Counter Screening and Secondary Assays. To counter-screen for inhibitors of the TNF pathway, HEK293-NFκB-Luc cells were seeded at 10⁵ cells per well in 90 μL medium in white 96-well plates (Greiner Bio-One) and cultured overnight, before adding compounds (5 μL in medium) to cells. After 2 h incubation, 5 μL TNF (200 ng/mL) (R&D Systems) was added (final concentration 10 ng/mL) and cells were incubated for 16 h. Luciferase activity was measured using Britelite kit (Perkin Elmer). To counter-screen for inhibitors of NF-κB induced by TLR4, CD40, NOD1, NOD2, cIAP2/MALT, or XIAP/TAB, the 293-NF-κB-luc cells cultured in 96 well plates as above were pretreated with compounds for 2 h and then transfected using Lipofectamine 2000 with various plasmids including pcDNA3 (“empty vector” control) or plasmids encoding CD4-TLR4, CD40, NOD1, NOD2, cIAP2/MALT, XIAP/TAB, using 0.2 μL of transfection reagent containing 100 ng DNA per well. Cells were cultured in medium containing CID-2858522 or other compounds and luciferase activity was measured 48 h later. Alternatively, 293-NF-κB-luciferase cells were cultured with 16 μM all-trans-retinoic acid for 48 hrs or 2 μM doxorubicin for 48 hrs before measuring luciferase reporter gene activity. The counter screen for inhibitors of luciferase was performed in 96 well white plates (Greiner Bio-one) containing 45 μL per well of ATPlite solution and luciferase (Perkin Elmer). Compounds diluted in 5 μL phosphate-buffered saline (PBS) were added at 8 μM final concentration. Reactions were then initiated by addition of 50 μL 160 nM ATP (Sigma) in PBS and luciferase activity was measured 2 h later using a luminometer (LJL Biosystems, Sunnyvale, Calif.).

Cell viability assay. Cell viability was estimated based on cellular ATP levels, measured using ATPlite kit (Perkin Elmer). Cells at a density of 10⁵/mL were seeded at 90 μL per well in 96-well white plates and cultured overnight. Compounds were added (5 μL in medium) to wells and cells were cultured for 16 h, Finally, 50 μL ATPlite solution was added to each well and luminescence activity was read using a luminometer (LJL Biosystems, Sunnyvale, Calif.).

Lymphokine measurements. Human IL-2 or IL-8 levels in culture medium were measured by Enzyme-Linked Immunosorbent Assays (ELISAs), using BD OptEIA ELISAs (BD Biosciences, San Diego, Calif.), according to the manufacturer's protocol, using 96-well ELISA plates (BD Biosciences) and measuring absorbance within 30 minutes of initiating reactions using a SpectraMax 190 spectrophotometer (Molecular Devices).

Dual-luciferase assay for NF-κB activity. Cells seeded in 96 well black plates were co-transfected with Renilla luciferase plasmid and NF-κB-responsive firefly luciferase reporter gene plasmid, with pcDNA3 control or plasmids encoding various desired proteins, using Lipofectamine 2000. The culture medium was aspirated and cells were washed with PBS, prior to adding 50 μL per well of Passive Lysis Buffer (Promega), followed by addition of Dual-luciferase assay reagent (Promega) and measurement of firefly and renilla luciferase activity, using a spectrofluorimeter.

NF-κB DNA-binding activity assays. Nuclear extracts were prepared from 10 cm² plates of confluent cells using a kit (Active Motif, Carsbad, Calif.). The total protein content of nuclear fractions was quantified by the Bradford method, followed by storage at −80° C. NF-κB DNA-binding activity was measured in nuclear extracts (10 μg protein) using an immunoassay method (TransAM Kit [Active Motif]) employing 96 well plates coated with double-strand oligodeoxynucleotides containing NF-κB consensus binding site (5′-GGGACTTTCC-3′) and anti-p65 antibody, which was detected by secondary horseradish peroxidase (HRP)-conjugated antibody, using a calorimetric substrate with absorbance read at 450 nm within 5 minutes using a spectrophotometer, SpectraMax M5 (Molecular Devices, Sunnyvale, Calif.).

Lymphocyte proliferation assay. Splenocytes were isolated from normal Balb/c mice and red blood cells were removed using a mouse erythrocyte lysis kit (R&D Systems, Minneapolis, Minn.). Splenocytes were suspended in RMPI-1640 medium supplemented with 10% FBS, 1% penicillin-streptomycin, and 1 mM L-glutamine. Cells were diluted into 2×10⁶ cells/mL and 200 μL were seeded in round bottom 96-well plates and incubated at 37° C. in 5% CO₂ and 95% relative humidity. Cells were pretreated with compounds or DMSO diluted in medium for 2 h, then treated with anti-CD3/anti-CD28 or anti-IgM antibodies for 48 h, prior to adding 1 μCi [³H]-Thymidine for (MP Biomedical, Solon, Ohio) 12 h. Cells were transferred to fiberglass filters (Wallac, Turku, Finland) using a FilterMate Harvester (Perkin Elmer), dried, and [³H]-incorporation into DNA was quantified by scintillation counting Betaplate Scint (Perkin Elmer) and a MicroBetaTrilux LCS and luminescence counter (Perkin Elmer).

Chronic Lymphocytic Leukemia (CLL) cell culture. Peripheral blood mononuclear cells from CLL patients were obtained under IRB approval from whole blood by Ficoll density gradient centrifugation and cultured with RPMI 1640 Medium supplemented with 10% FBS and antibiotics.

In Vitro Kinase Assays. PKC-beta, PKC-theta and IKK-beta in vitro kinase assays were performed using the HTScan Kinase Assay Kit (Cell Signaling, Danvers, Mass.) according to manufacturer's protocols. A panel of >300 kinases was screened by Ambit, Inc.

Several compounds synthesized by the methods described herein were tested for their efficacy at inhibiting Nf-κB activation. The results are set forth in Tables I and II, below.

TABLE I
Nf-κB inhibition of selected tertiary amine compounds

| Structure | Compound | R₁ | IC₅₀ |
|---|---|---|---|
| 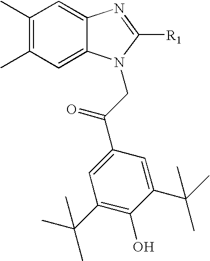 | 12 13 14 15 16 17 18 19 20 21 22 23 24 25 26 27 28 29 30 31 | piperdine morpholine pyrrollidine (S)-2(methoxymethyl)pyrrollidine (S)-1-(2-Pyrrolidinylmethyl)pyrrolidine 2,4-dimethyl-3-ethylpyrrollidine 3-hydroxypyrrollidine 2-(hydroxymethyl)piperidine 4-benzylpiperidine 3-methylpiperidine 4-hydroxypiperidine 2-methylpiperidine 3,5-dimethylpiperidine 4-methylpiperidine N,N-dimethyl-1-piperidin-2-ylmethanamine N-methylpiperazine N-ethylpiperazine N-benzylpiperazine N-hydroxyethylpiperazine N-(2-methoxyphenyl)piperazine | >5 μM >5 μM >5 μM >5 μM >5 μM >5 μM >5 μM >5 μM >5 μM >5 μM >5 μM >5 μM >5 μM >5 μM >5 μM >5 μM >5 μM >5 μM >5 μM >5 μM |

TABLE II
Inhibition of antigen receptor-mediated Nf-κB

| Structure | Cmpd | R₁ | R₂ | R₃ | R₄ | IC₅₀ |
|---|---|---|---|---|---|---|
| 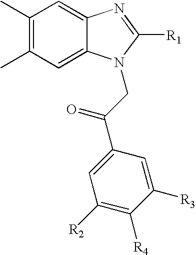 | 1 32 33 34 35 36 37 38 39 40 41 | 3-hydroxypropylamino 3-hydroxypropylamino 3-methoxypropylamino 3-methoxypropylamino 3-methoxypropyl-N-methylamino 3-hydroxypropylamino 3-methoxypropylamino 3-hydroxypropylamino 3-methoxypropylamino n-butylamino propargylamino | t-butyl t-butyl t-butyl t-butyl t-butyl methyl methyl H H t-butyl t-butyl | t-butyl t-butyl t-butyl t-butyl t-butyl methyl methyl H H t-butyl t-butyl | OH OMe OH OMe OH OH OH OH OH OH OH | 0.07 μM 0.07 μM 0.25 μM 0.25 μM 2.5 μM >5 μM >5 μM >5 μM >5 μM 0.1 μM >8 μM |

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A compound of Formula I or Formula II 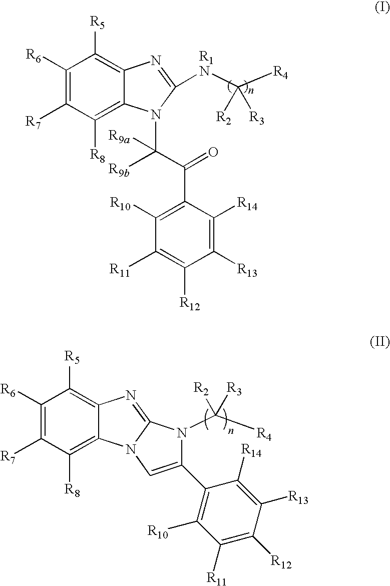
or a pharmaceutically acceptable salt or prodrug thereof, where
R₁ is selected from the group consisting of hydrogen, alkyl, cycloalkyl, and aryl;
R₂, R₃, R₉ₐ, and R_{9b} are each independently selected from the group consisting of hydrogen, alkyl, cycloalkyl, and aryl;
R₄ is selected from the group consisting of hydrogen, —OR₂₀, —SR₂₀, —C(O)OR₂₀, and —C(O)R₂₀;
R₅-R₈ and R₁₀-R₁₄ are each independently selected from the group consisting of hydrogen, —OR₂₀, —SR₂₀, —C(O)OR₂₀, —C(O)R₂₀, alkyl, cycloalkyl, and aryl;
R₂₀ is selected from the group consisting of hydrogen, alkyl, cycloalkyl, and aryl; and
n is an integer between 0-10.

2. The compound of claim 1, wherein R₁ is selected from the group consisting of hydrogen, methyl, ethyl, propyl, n-butyl, and t-butyl.

3. The compound of claim 1, wherein R₂ and R₃ are each independently selected from the group consisting of hydrogen, methyl, ethyl, propyl, n-butyl, and t-butyl.

4. The compound of claim 1, wherein R₉ₐ and R_{9b} are each independently selected from the group consisting of hydrogen, methyl, ethyl, propyl, n-butyl, and t-butyl.

5. The compound of claim 1, wherein R₅-R₈ are each independently selected from the group consisting of hydrogen, methyl, ethyl, propyl, n-butyl, and t-butyl.

6. The compound of claim 1, wherein R₄ is selected from the group consisting of hydrogen, —OR₂₀, —SR₂₀, —C(O)OR₂₀, and —C(O)R₂₀; wherein R₂₀ is selected from the group consisting of hydrogen, methyl, ethyl, propyl, n-butyl, t-butyl, and phenyl.

7. The compound of claim 1, wherein R₄ is selected from the group consisting of —OH, —OCH₃, and —OPh.

8. The compound of claim 1, wherein R₅ and R₈ are hydrogen.

9. The compound of claim 1, wherein R₆ and R₇ are methyl.

10. The compound of claim 1, wherein R₁₀ and R₁₄ are each independently selected from the group consisting of hydrogen, methyl, ethyl, propyl, n-butyl, and t-butyl.

11. The compound of claim 1, wherein R₁₁ and R₁₃ are each independently selected from the group consisting of hydrogen, methyl, ethyl, propyl, n-butyl, and t-butyl.

12. The compound of claim 1, wherein R₁₂ is selected from the group consisting of hydrogen, —OR₂₀, —SR₂₀, —C(O)OR₂₀, —C(O)R₂₀, methyl, ethyl, propyl, n-butyl, and t-butyl, wherein R₂₀ is selected from the group consisting of hydrogen, methyl, ethyl, propyl, n-butyl, t-butyl, and phenyl.

13. The compound of claim 1, wherein n is an integer between 1-5.

14. The compound of claim 1, wherein the compound has the structure:

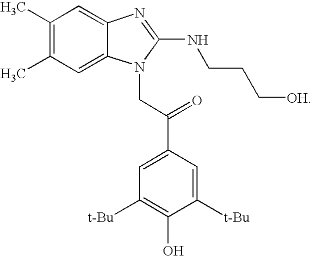

15. A pharmaceutical composition comprising a compound of Formula I or Formula II 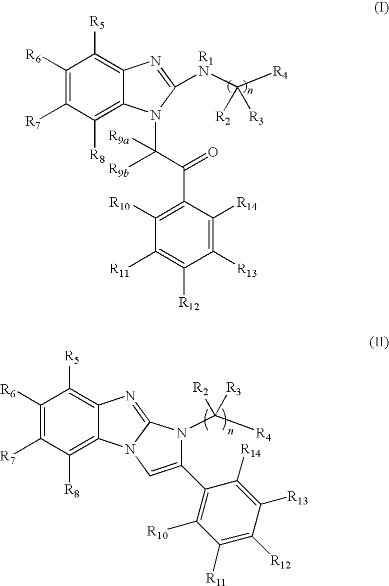
or a pharmaceutically acceptable salt or prodrug thereof, and a pharmaceutically acceptable diluent, excipient, or carrier, where
R₁ is selected from the group consisting of hydrogen, alkyl, cycloalkyl, and aryl;
R₂, R₃, R₉ₐ, and R_{9b} are each independently selected from the group consisting of hydrogen, alkyl, cycloalkyl, and aryl;
R₄ is selected from the group consisting of hydrogen, —OR₂₀, —SR₂₀, —C(O)OR₂₀, and —C(O)R₂₀;
R₅-R₈ and R₁₀-R₁₄ are each independently selected from the group consisting of hydrogen, —OR₂₀, —SR₂₀, —C(O)OR₂₀, —C(O)R₂₀, alkyl, cycloalkyl, and aryl;
R₂₀ is selected from the group consisting of hydrogen, alkyl, cycloalkyl, and aryl; and
n is an integer between 0-10.

16. A method of identifying a compound that selectively inhibits antigen receptor-mediated NF-κB activation comprising:
(a) providing an aqueous solution comprising a cell transfected with a reporter gene driven by a NF-κB responsive promoter;
(b) adding to the solution a test compound;
(c) adding to the solution an NF-κB inducing stimulus; and
(d) determining whether the test compound reduces the cell response to the stimulus;
wherein the test compound is a compound of Formula I or Formula II 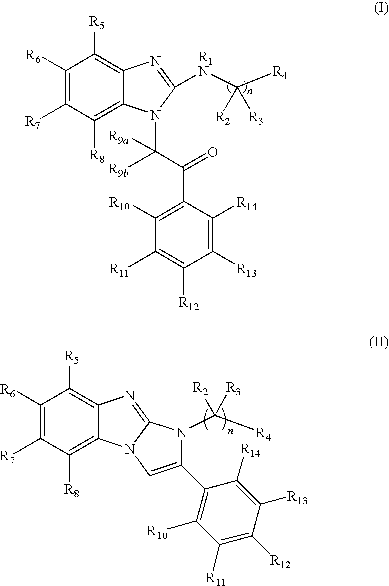
or a pharmaceutically acceptable salt or prodrug thereof, where
R₁ is selected from the group consisting of hydrogen, alkyl, cycloalkyl, and aryl;
R₂, R₃, R₉ₐ, and R_{9b} are each independently selected from the group consisting of hydrogen, alkyl, cycloalkyl, and aryl;
R₄ is selected from the group consisting of hydrogen, —OR₂₀, —SR₂₀, —C(O)OR₂₀, and —C(O)R₂₀;
R₅-R₈ and R₁₀-R₁₄ are each independently selected from the group consisting of hydrogen, —OR₂₀, —SR₂₀, —C(O)OR₂₀, —C(O)R₂₀, alkyl, cycloalkyl, and aryl;
R₂₀ is selected from the group consisting of hydrogen, alkyl, cycloalkyl, and aryl; and
n is an integer between 0-10.

17. The method of claim 16, wherein the reporter gene is a luciferase reporter gene driven by a NF-κB responsive promoter.

18. The method of claim 16, wherein the test compound reduces the response to the stimulus by greater than 50 percent.

19. A method of selectively inhibiting antigen receptor-mediated NF-κB activation in a cell comprising contacting the cell with a compound of Formula I or Formula II 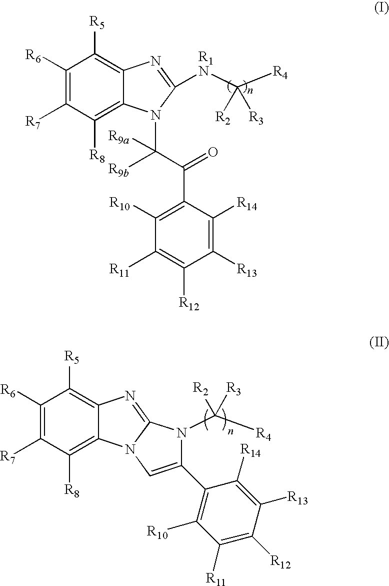
or a pharmaceutically acceptable salt or prodrug thereof, where
R₁ is selected from the group consisting of hydrogen, alkyl, cycloalkyl, and aryl;
R₂, R₃, R₉ₐ, and R_{9b} are each independently selected from the group consisting of hydrogen, alkyl, cycloalkyl, and aryl;
R₄ is selected from the group consisting of hydrogen, —OR₂₀, —SR₂₀, —C(O)OR₂₀, and —C(O)R₂₀;
R₅-R₈ and R₁₀-R₁₄ are each independently selected from the group consisting of hydrogen, —OR₂₀, —SR₂₀, —C(O)OR₂₀, —C(O)R₂₀, alkyl, cycloalkyl, and aryl;
R₂₀ is selected from the group consisting of hydrogen, alkyl, cycloalkyl, and aryl; and
n is an integer between 0-10.

20. The method of claim 30, wherein the contacting is in vivo or in vitro.

21. A method of selectively inhibiting antigen receptor-mediated NF-κB activation in a subject comprising identifying a subject in need thereof and administering to the subject, or contacting the subject with, a compound of Formula I or Formula II 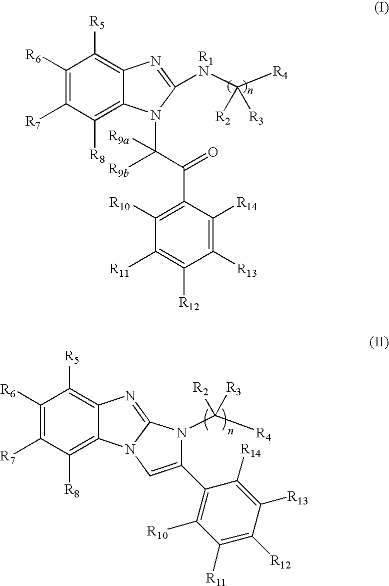
or a pharmaceutically acceptable salt or prodrug thereof, where
R₁ is selected from the group consisting of hydrogen, alkyl, cycloalkyl, and aryl;
R₂, R₃, R₉ₐ, and R_{9b} are each independently selected from the group consisting of hydrogen, alkyl, cycloalkyl, and aryl;
R₄ is selected from the group consisting of hydrogen, —OR₂₀, —SR₂₀, —C(O)OR₂₀, and —C(O)R₂₀;
R₅-R₈ and R₁₀-R₁₄ are each independently selected from the group consisting of hydrogen, —OR₂₀, —SR₂₀, —C(O)OR₂₀, —C(O)R₂₀, alkyl, cycloalkyl, and aryl;
R₂₀ is selected from the group consisting of hydrogen, alkyl, cycloalkyl, and aryl; and
n is an integer between 0-10.

22. A method of treating a disease associated with antigen receptor-mediated NF-κB activation in a subject comprising
identifying a subject in need thereof and administering to the subject, or contacting the subject with, a compound of Formula I or Formula II 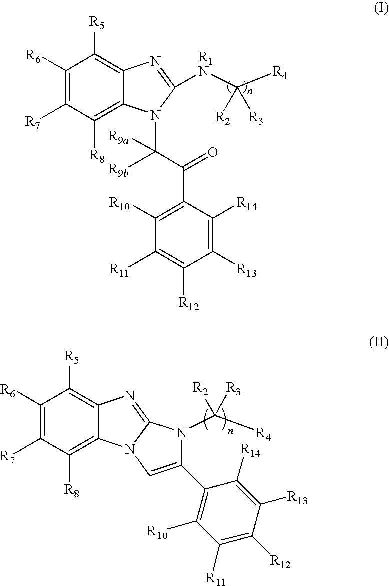
or a pharmaceutically acceptable salt or prodrug thereof, where
R₁ is selected from the group consisting of hydrogen, alkyl, cycloalkyl, and aryl;
R₂, R₃, R₉ₐ, and R_{9b} are each independently selected from the group consisting of hydrogen, alkyl, cycloalkyl, and aryl;
R₄ is selected from the group consisting of hydrogen, —OR₂₀, —SR₂₀, —C(O)OR₂₀, and —C(O)R₂₀;
R₅-R₈ and R₁₀-R₁₄ are each independently selected from the group consisting of hydrogen, —OR₂₀, —SR₂₀, —C(O)OR₂₀, —C(O)R₂₀, alkyl, cycloalkyl, and aryl;
R₂₀ is selected from the group consisting of hydrogen, alkyl, cycloalkyl, and aryl; and
n is an integer between 0-10.
